(19) United States
(12) Patent Application Publication
(10) Pub. No.: US 2026/0124197 A1
(43) Pub. Date: May 7, 2026

(54) NOVEL HETEROBICYCLIC COMPOUND FOR INHIBITING YAP-TEAD INTERACTION AND PHARMACEUTICAL COMPOSITION COMPRISING SAME

(71) Applicant: HANMI PHARM. CO., LTD., Hwaseong-si, Gyeonggi-do (KR)

(72) Inventors: Ji Sook KIM, Hwaseong-si (KR); Joo Yun BYUN, Hwaseong-si (KR); Kwee Hyun SUH, Hwaseong-si (KR); Young Gil AHN, Hwaseong-si (KR); Ji Hee YOON, Hwaseong-si (KR); Young Kyo JEON, Hwaseong-si (KR); Seung Hyun JUNG, Hwaseong-si (KR); Seon Yeong HAN, Hwaseong-si (KR)

(21) Appl. No.: 19/120,670

(22) PCT Filed: Oct. 12, 2023
(86) PCT No.: PCT/KR2023/015758

§ 371 Date: Apr. 11, 2025

(30) Foreign Application Priority Data
Oct. 13, 2022 (KR) 10-2022-0131762

(51) Int. Cl. A61K 31/4965; A61K 31/404; A61K 31/4155; A61K 31/4178; A61K 31/4188; A61K 31/422; A61K 31/437; A61K 31/4439; A61P 35/00; C07D 209/10; C07D 401/04; C07D 401/14; C07D 403/04; C07D 405/04; C07D 405/14; C07D 409/04; C07D 413/04; C07D 471/04; C07D 487/04

(52) U.S. Cl. CPC A61K 31/4965; A61K 31/404; A61K 31/4155; A61K 31/4178; A61K 31/4188; A61K 31/422; A61K 31/437; A61K 31/4439; A61P 35/00; C07D 209/10; C07D 401/04; C07D 401/14; C07D 403/04; C07D 405/04; C07D 405/14; C07D 409/04; C07D 413/04; C07D 471/04; C07D 487/04

(57) ABSTRACT

Provided are a compound, selected from compounds of Formula 1, enantiomers, diastereomers, solvates and hydrates thereof, and pharmaceutically acceptable salts thereof, methods of preparing the same, and use thereof.

### TECHNICAL FIELD

The present disclosure relates to a pharmaceutical composition including a heterobicyclic compound that inhibits Yes associated protein (YAP)-transcriptional enhancer associate domain (TEAD) binding, wherein the compound according to the present disclosure may directly inhibit YAP-TEAD binding in the Hippo pathway, which plays a key role in the development of cancer.

### BACKGROUND ART

A Hippo signaling cascade is an important pathway for cancer biogenesis and tumor maintenance. YAP and tafazzin (TAZ) are transcriptional co-activators of a Hippo pathway network and regulate cell proliferation, migration, and apoptosis. Inactivation of a Hippo signaling pathway promotes YAP/TAZ translocation to a nucleus, where YAP/TAZ interacts with transcriptional enhancer associate domain (TEAD) transcription factors and co-activates an expression of target genes and promotes cell proliferation. Target genes such as connective tissue growth factor (CTGF) and Cyr61, AXL receptor tyrosine kinase, and MYC, which are strongly correlated with tumor development, are regulated by TEAD. TEAD has also been shown to be overexpressed in breast cancer stem cells and breast cancer, ovarian cancer, germ cell tumor, renal cell carcinoma, medulloblastoma, and gastric cancer, etc. Overactivation of YAP and TAZ and/or mutations in one or more members of the Hippo pathway network have been associated with numerous cancers. In addition, recent studies have reported that resistance to EGFR tyrosine kinase inhibitors Tarceva (erlotinib), Iressa (gefitinib) or Tagrisso (osimertinib) is associated with YAP overexpression or amplification along with epithelial-mesenchymal transition (EMT) phenotypic changes.

The inventors of the present disclosure have completed the present disclosure by developing a novel heterobicyclic compound for the inhibition of YAP-TEAD protein interactions.

### PRIOR ARTS

### Patent Documents

(Patent Document 1) International Publication No. WO2019/040380
(Patent Document 2) International Publication No. WO2020/243415

### Non-Patent Documents

(Non-patent Document 1) Semin. Cancer Biol. 2022, 85, 33
(Non-patent Document 2) Nat. Rev. Drug Discov. 2014, 13 (1), 63
(Non-patent Document 3) Cancer Res. 2011, 71 (3), 873
(Non-patent Document 4) J. Cell Mol. Med. 2017, 21 (11), 2663
(Non-patent Document 5) Cancer Cell 2020, 37, 104
(Non-patent Document 6) Cells 2021, 10, 2715
(Non-patent Document 7) Genes Cancer 2017, 8 (3-4), 497

### DISCLOSURE OF INVENTION

### Technical Problem

An objective of the present disclosure is to provide a novel heterobicyclic compound with high inhibitory activity against YAP-TEAD binding in the Hippo pathway, which plays a key role in the development of cancer.

Another objective of the present disclosure is to provide a pharmaceutical composition including the aforementioned compound as an active ingredient for treating or preventing dysregulation of the Hippo signaling pathway, specifically related diseases caused by TEAD activation.

Other objectives and advantages of the present application will become apparent from the following detailed description in conjunction with the appended claims. Anything not set forth herein may be readily recognized and inferred by one of ordinary skill in the art of the present application or a similar art and is hereby omitted.

### Solution to Problem

According to an embodiment of the present disclosure, provided is a compound selected from compounds of Formula 1 below, enantiomers, diastereomers, solvates and hydrates thereof, and pharmaceutically acceptable salts thereof.

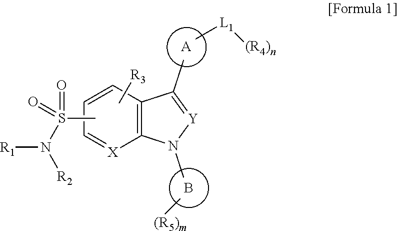

According to an embodiment of the present disclosure, provided is a pharmaceutical composition for treating or preventing dysregulation of the Hippo signaling pathway, specifically related diseases caused by TEAD activation, the pharmaceutical composition including, as an active ingredient, the compound selected from the compounds of Formula 1, the enantiomers, diastereomers, solvates and hydrates thereof, and the pharmaceutically acceptable salts thereof.

### Advantageous Effects of Invention

A novel heterobicyclic compound that has a structure of Formula 1 of the present disclosure has excellent inhibitory activity against YAP-TEAD binding, and may be useful as a therapeutic agent for a number of diseases involving the Hippo pathway, which plays a key role in the development of cancer.

### MODE FOR THE INVENTION

The following describes the present disclosure in more detail.

All technical terms used in the present disclosure, unless otherwise defined, are used as commonly understood by one of ordinary skill in the relevant field of the present disclosure. In addition, while suitable methods or samples are described herein, similar or equivalent methods are also included within the scope of the present disclosure.

According to an embodiment of the present disclosure, provided is a compound selected from compounds of Formula 1 below, enantiomers, diastereomers, solvates and hydrates thereof, and pharmaceutically acceptable salts thereof.

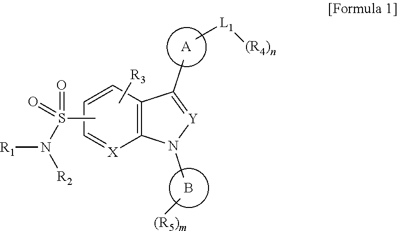

In Formula 1 above,
R₁ and R₂ may each independently be hydrogen, C₁₋₆alkyl, C₂₋₆alkenyl, C₃₋₆carbocyclyl, or haloC₁₋₆alkyl;
R₅ may be hydrogen, halogen, C₁₋₆alkyl, haloC₁₋₆alkyl, C₁₋₆alkoxy, or cyano;
\textcircled{A} may be carbocyclyl or heterocyclyl;
\textcircled{B} may be C₃₋₁₀aryl or C₄₋₁₀heteroaryl;
L₁ may be absent, bonded, or C₁₋₃alkylene, or C₁₋₃alkylene substituted with a halogen;
each R₄ and each R₅ may independently be hydrogen, halogen, cyano, amino, C₁₋₆alkyl, haloC₁₋₆alkyl, C₁₋₆alkoxy, C₁₋₆alkoxyalkyl, haloC₁₋₆alkoxy, mono-(C₁₋₃alkyl)-substituted carbamoyl (—(CO)—NH(C₁₋₃alkyl)), di-(C₁₋₃alkyl)-substituted carbamoyl (—(CO)—N(C₁₋₃alkyl) 2), C₁₋₃alkylsulfinyl (—(SO)—(C₁₋₃alkyl)), C₁₋₃alkylsulfonyl (—SO₂—(C₁₋₃alkyl)), substituted or unsubstituted C₃₋₆carbocyclyl, substituted or unsubstituted C₆₋₁₀aryl, substituted or unsubstituted C₂₋₆heterocyclyl, or substituted or unsubstituted C₄₋₁₀heteroaryl;
X and Y may each independently be —C— or —N—; and
m and n may each independently be an integer from 0 to 3.
As used herein, the term “halogen” may be F, Cl, Br, or I.

As used herein, unless otherwise noted, the term “alkyl” refers to a straight-chain or branched hydrocarbon residue, which may be substituted or unsubstituted. The alkyl group may be, for example, methyl, ethyl, propyl, butyl, pentyl, isopropyl, isobutyl, or t-butyl, but is not limited thereto.

As used herein, the term “alkylene” refers to a divalent straight-chain or branched hydrocarbon group having (—CH₂—)ₚ. p may be any integer.

As used herein, unless otherwise noted, the term “alkenyl” refers to an alkyl group including one or more double bonds, which may be substituted or unsubstituted. The alkenyl group may be, for example, prop-1-ene, but-1-ene, but-2-ene, 3-methylbut-1-ene, or pent-1-ene, etc., but is not limited to.

As used herein, unless otherwise noted, the term “cycloalkyl” refers to saturated monocyclic and polycyclic hydrocarbon rings typically having a stated number of carbon atoms, including rings which may be substituted or unsubstituted. The cycloalkyl group may be, for example, cyclopropyl, cyclobutyl, cyclopentyl, cyclohexyl, or cycloheptyl, etc., but is not limited thereto.

As used herein, unless otherwise noted, the term “heterocycloalkyl” refers to a monocyclic, which may be substituted or unsubstituted, cyclic alkyl including one or more heteroatoms selected from N, O, and S. The heterocycloalkyl group may be, for example, piperidinyl, piperazinyl, morpholinyl, pyrrolidinyl, thiomorpholinyl, imidazolidinyl, tetrahydrofuranyl, or similar groups, etc., but is not limited thereto.

As used herein, unless otherwise noted, the term “haloalkyl” refers to include monohaloalkyl and polyhaloalkyl, which may be substituted or unsubstituted. The terms halogen and alkyl are as defined above.

As used herein, unless otherwise noted, the term “alkoxy” refers to a straight-chain or branched hydrocarbon residue, which may be substituted or unsubstituted, connected by an oxygen. The alkoxy may be, for example, methoxy, ethoxy, propoxy, butoxy, isopropoxy, isobutoxy, or t-butoxy, etc., but is not limited thereto.

As used herein, the term “alkoxyalkyl” refers to an alkyl in which one or more hydrogen atoms of the alkyl group are substituted with an alkoxy. The alkoxyalkyl may be, for example, methoxymethyl, ethoxymethyl, methoxyethyl, ethoxyethyl, methoxypropyl, ethoxypropyl, and isopropoxymethyl, etc., but is not limited thereto.

As used herein, unless otherwise noted, the term “aryl” refers to an aromatic group which may be substituted or unsubstituted, and may be including, for example, C₃₋₁₀aryl, C₃₋₈aryl, or C₃₋₆aryl, with double bonds alternating (resonating) between adjacent carbon atoms or suitable heteroatoms. For example, may be, phenyl, biphenyl, naphthyl, toluyl, or naphthalenyl, etc., but is not limited thereto.

As used herein, unless otherwise noted, the term “heteroaryl” may refer to any aromatic group, monocyclic or bicyclic, which may be substituted or unsubstituted, that includes one or more heteroatoms selected from N, O, and S. For example, a monocyclic heteroaryl may be, pyridinyl, imidazolyl, thiazolyl, oxazolyl, thiophenyl, furanyl, pyrrolyl, isoxazolyl, pyrazolyl, triazolyl, thiadiazolyl, tetrazolyl, oxadiazolyl, pyridazinyl, pyrimidinyl, or pyrazinyl, etc., but is not limited thereto. For example, a bicyclic heteroaryl may be, indolyl, benzothiophenyl, benzofuranyl, benzimidazolyl, benzoxazolyl, benzisoxazolyl, benzthiazolyl, benzthiadiazolyl, benztriazolyl, quinolinyl, isoquinolinyl, purinyl, or pyropyridinyl, etc., but is not limited thereto.

As used herein, unless otherwise noted, the term “carbocyclyl” refers to a substituent including a carbon ring atom that has the structure of a saturated carbocyclyl (for example, “cycloalkyl”), a partially saturated carbocyclyl (for example, “cycloalkenyl”), or a completely unsaturated carbocyclyl (for example, “aryl”). The carbocyclyl may be single ring (monocyclic) or polycyclic ring structured. As used herein, a carbocyclyl includes, for example, 3 to 14 carbon ring atoms, or, for example, 3 to 8 carbon ring atoms, and may be saturated, unsaturated, or aromatized. Herein, ring atoms are atoms that are bonded together to form a ring or rings of the carbocyclyl substituent. For example, a saturated carbocyclyl group may be, cyclopropyl, cyclopentyl, or cyclohexyl, etc., but is not limited thereto. For example, unsaturated carbocyclyl may include three or less double bonds. For example, an aromatic carbocyclyl group may be phenyl. In addition, the term “carbocyclyl” may include fused combinations of carbocyclyl groups, and for example, may be naphthyl, phenanthryl, indanyl, and indenyl, etc., but is not limited thereto.

As used herein, unless otherwise noted, the term “heterocyclyl” refers to a substituent including at least one heteroatom with a ring atom, that has the structure of a saturated heterocyclyl (for example, “heterocycloalkyl”), a partially saturated heterocyclyl (for example, “heterocycloalkenyl”), or a completely unsaturated heterocyclyl (for example, “heteroaryl”). The heterocyclyl may be single ring (monocyclic) or polycyclic ring structured. As used herein, a heterocyclyl includes, for example, a total of 3 to 14 ring atoms, 6 to 14 ring atoms, or, for example, a total of 3 to 8 ring atoms, and may be saturated, unsaturated, or aromatized. Herein, ring atoms are atoms that are bonded together to form a ring or rings of the heterocyclyl substituent. For example, at least one of the ring atoms is nitrogen, oxygen, or sulfur, and the remaining ring atoms are independently selected from the group consisting of carbon, nitrogen, oxygen, and sulfur. For example, the ring atoms of the heterocyclyl may include 4 or less heteroatoms such as N, O, and S, for example, a total of 3 to 14 ring atoms, or for example, a total of 5 to 7 ring atoms, and may be saturated, unsaturated, or aromatized. For example, the heterocyclyl may be, furanyl, thiophenyl, pyrrolyl, pyrrolinyl, pyrrolidinyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, tetrazolyl, thiadiazolyl, pyranyl, pyridinyl, piperidinyl, dioxanyl, morpholinyl, dithianyl, thiomorpholinyl, pyridazinyl, pyrimidinyl, pyrazinyl, piperazinyl, sulfolanyl, triazinyl, azepinyl, oxazepinyl, thiazepinyl, diazepinyl, or thiazolinyl, but is not limited thereto. In addition, the term heterocyclyl may include fused heterocyclyl groups, for example, may be benzimidazolinyl, benzoxazolyl, imidazopyridinyl, benzoxazinyl, benzothiazinyl, oxazolopyridinyl, quinolinyl, quinazolinyl, quinoxazolinyl, dihydroquinazolinyl, benzothiazolyl, phthalimidyl, benzofuranyl, benzodiazepinyl, indolyl, or isoindolyl, but is not limited thereto. “Heterocyclyl” may be a carbon linker or a heteroatom linker. For example, N-linked heterocyclyl of heteroatomic linkers include,

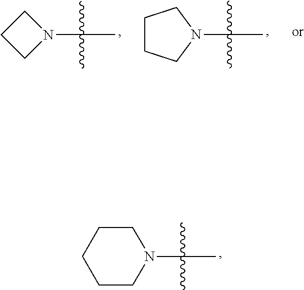 [SMILES: *N1CCC1 ; *N1CCCC1 ; *N1CCCCC1]

but is not limited thereto. As used herein, unless otherwise noted, the term “fused heteroaryl” refers to a connected, substituted or unsubstituted ring system in which a heteroaryl group is fused to another aryl, heteroaryl or heterocycloalkyl group. For example, a fused heteroaryl may form a 5+5-membered, 5+6-membered, 5+7-membered, 6+6-membered, or 6+7-membered fused ring system. In addition, the fused heteroaryl may be, for example,

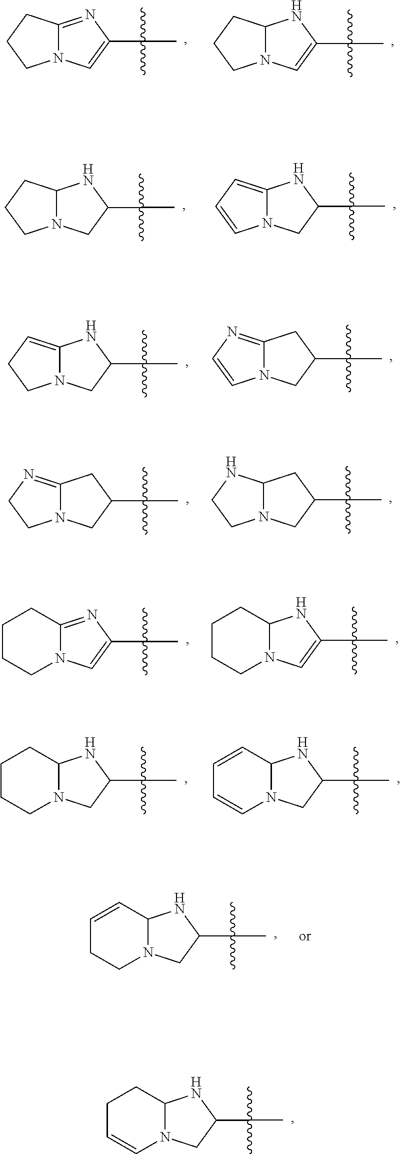

etc., but is not limited thereto.

As used herein, any substituent may be any one substituent selected from, for example, cyano, amino, hydroxy, C₁₋₆alkyl, haloC₁₋₆alkyl, C₁₋₆alkoxy, and haloC₁₋₆alkoxy, but is not limited thereto.

For example, substituted C₃₋₆carbocyclyl, C₆₋₁₀aryl, C₂₋₆heterocyclyl, C₄₋₁₀heteroaryl, C₃₋₆cycloalkyl, or C₂₋₆heterocycloalkyl may be substituted with any one substituent selected from one or more hydrogen atoms of an alkyl group by halogen, cyano, amino, hydroxy, C₁₋₆alkyl, haloC₁₋₆alkyl, C₁₋₆alkoxy, and haloC₁₋₆alkoxy, but is not limited thereto.

As used herein, the term “stereoisomer” may refer to compounds of the present disclosure or salts thereof that have the same chemical formula or molecular formula but differ optically or sterically, and may include enantiomers or diastereomers.

As used herein, the term “enantiomer” refers to two stereoisomers of a compound that are non-overlapping mirror images of each other.

As used herein, the term “diastereomer” refers to a stereoisomer with two or more chiral centers, the molecules of which are not mirror images of each other.

The compounds of the present disclosure may include asymmetric or chiral centers, and thus may exist in different stereoisomeric forms. All stereoisomeric forms of the compounds of the present disclosure, such as diastereomers, enantiomers, and racemic mixtures, are considered to compose a portion of the present disclosure. A 50:50 mixture of enantiomers is referred to as a racemic mixture or racemate.

As used herein, the term “solvate” may refer to a compound of the present disclosure or a salt thereof including a stoichiometric or non-stoichiometric amount of solvent bound by non-covalent intermolecular forces. Suitable solvents may include those that are volatile, non-toxic, and/or suitable for human administration. A “solvate” may include a molecular complex including the compound and one or more pharmaceutically acceptable solvent molecules, for example ethanol.

As used herein, the term “hydrate” refers to a complex in which the solvent molecule is water.

As used herein, the term “pharmaceutically acceptable salt” refers to a pharmaceutically acceptable organic or inorganic salt, which may be prepared by any suitable method useful to those skilled in the art. For example, when the compound of the present disclosure is a base, the desired pharmaceutically acceptable salt may be prepared by any suitable method useful to those skilled in the art, for example, by treating the free base with an inorganic acid or organic acid.

In an embodiment, \textcircled{A} is C₆₋₁₀aryl, C₁₋₁₀heteroaryl, Con fused heteroaryl, or C₂₋₆heterocyclyl,
wherein C₁₋₁₀ heteroaryl, C₆₋₁₄ fused heteroaryl, or C₂₋₆ heterocyclyl may include 1 to 4 heteroatoms each independently selected from N, O, and S.

In an embodiment, \textcircled{A} may be a phenyl group, a pyridinyl group, a pyrazinyl group, a pyrazolyl group, an imidazolyl group, a thiophenyl group, a furanyl group, an oxazole group, an azetidinyl group, or

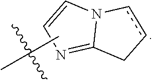

[symbol] refers to a single bond or a double bond.

For example, \textcircled{A} may be

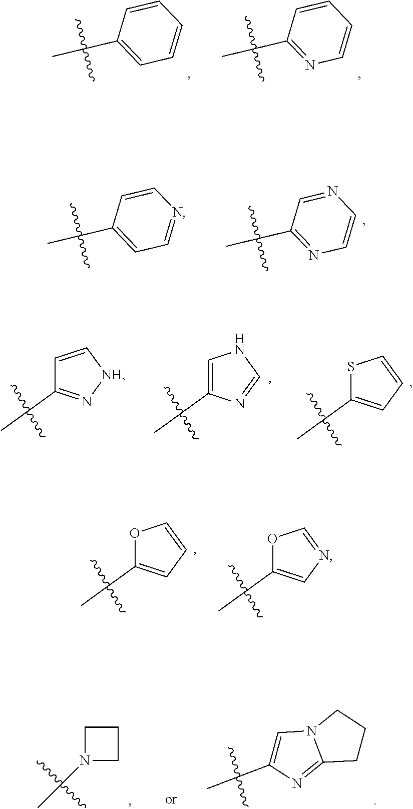

In an embodiment, \textcircled{B}, may be a phenyl group or a pyridinyl group.

In an embodiment, R₁ and R₂ may each independently be hydrogen, C₁₋₆alkyl, C₂₋₆alkenyl, C₃₋₆cycloalkyl, or haloC₁₋₆alkyl.

In an embodiment, L₁ is a bond; and

each of R₄ and each of R₅ may be independently selected from hydrogen, halogen, cyano, C₁₋₆alkyl, haloC₁₋₆alkyl, C₁₋₆alkoxy, C₁₋₆alkoxyalkyl, haloC₁₋₆alkoxy, mono-(C₁₋₃alkyl)-substituted carbamoyl (—(CO)—NH(C₁₋₃alkyl)), di-(C₁₋₃alkyl)-substituted carbamoyl (—(CO)—N(C₁₋₃alkyl)₂), C₁₋₃alkylsulfonyl (—SO₂—(C₁₋₃alkyl)), substituted or unsubstituted C₃₋₆cycloalkyl, substituted or unsubstituted C₆₋₁₀aryl, or substituted or unsubstituted C₂₋₆heterocycloalkyl.

In an embodiment, L₁ is C₁₋₃alkylene; and
each R₄ and each R₅ may be independently selected from hydrogen, halogen, cyano, C₁₋₆alkyl, haloC₁₋₆alkyl, C₁₋₆alkoxy, haloC₁₋₆alkoxy, di-(C₁₋₃alkyl)-substituted carbamoyl (—(CO)—N(C₁₋₃alkyl) 2), C₁₋₃alkylsulfonyl (—SO₂—(C₁₋₃alkyl)), substituted or unsubstituted C₃₋₆cycloalkyl, substituted or unsubstituted C₆₋₁₀aryl, or substituted or unsubstituted C₂₋₆heterocycloalkyl.

In an embodiment, R₅ may each be hydrogen, halogen, cyano, C₁₋₆alkyl, haloC₁₋₆alkyl, C₁₋₆alkoxy, haloC₁₋₆alkoxy, C₃₋₆cycloalkyl, or phenyl; or

C₃₋₆ cycloalkyl or phenyl substituted with any one or more substituents selected from halogen, cyano, C₁₋₆alkyl, haloC₁₋₆alkyl, C₁₋₆alkoxy, and haloC₁₋₆alkoxy.

In an embodiment, R₅ may each be hydrogen, halogen, cyano, methyl, ethyl, propyl, t-butyl, trifluoromethyl, trifluoromethoxy, cyclopropyl, cyclobutyl, cyclopentyl, cyclohexyl, or phenyl.

In an embodiment, the compound may be a compound selected from the following compounds, enantiomers, diastereomers, solvates and hydrates thereof, and pharmaceutically acceptable salts thereof.
1)N-methyl-3-(3-methylpyrazin-2-yl)-1-(4-(trifluoromethyl)phenyl)-1H-indole-5-sulfonamide;
2)N-methyl-3-(1-methyl-1H-imidazol-4-yl)-1-(4-(trifluoromethyl)phenyl)-1H-indole-5-sulfonamide;
3)N-methyl-3-(1-methyl-1H-pyrazol-3-yl)-1-(4-(trifluoromethyl)phenyl)-1H-indole-5-sulfonamide;
4)N-methyl-3-(1-methyl-1H-pyrazol-4-yl)-1-(4-(trifluoromethyl)phenyl)-1H-indole-5-sulfonamide;
5)N-methyl-3-(1-methyl-1H-imidazol-4-yl)-1-(4-(trifluoromethoxy)phenyl)-1H-indole-5-sulfonamide;
6) 3-(1-isopropyl-1H-imidazol-4-yl)-N-methyl-1-(4-(trifluoromethyl)phenyl)-1H-indole-5-sulfonamide;
7)N-ethyl-3-(1-methyl-1H-imidazol-4-yl)-1-(4-(trifluoromethyl)phenyl)-1H-indole-5-sulfonamide;
8)N-methyl-3-(pyridin-2-yl)-1-(4-(trifluoromethyl)phenyl)-1H-indole-5-sulfonamide;
9)N-methyl-3-(5-methylthiophen-2-yl)-1-(4-(trifluoromethyl)phenyl)-1H-indole-5-sulfonamide;
10)N-methyl-3-(5-methylfuran-2-yl)-1-(4-(trifluoromethyl)phenyl)-1H-indole-5-sulfonamide;
11) 3-(1-ethyl-1H-imidazol-4-yl)-N-methyl-1-(4-(trifluoromethyl)phenyl)-1H-indole-5-sulfonamide;
12)N-methyl-3-(1-methyl-1H-imidazol-4-yl)-1-(5-(trifluoromethyl)pyridin-2-yl)-1H-indole-5-sulfonamide;
13) 3-(2-fluorophenyl)-N-methyl-1-(4-(trifluoromethyl)phenyl)-1H-indole-5-sulfonamide;
14) 1-(4-chlorophenyl)-N-methyl-3-(1-methyl-1H-imidazol-4-yl)-1H-indole-5-sulfonamide;
15)N-methyl-3-(pyridin-4-yl)-1-(4-(trifluoromethyl)phenyl)-1H-indole-5-sulfonamide;
16) 3-(1-cyclobutyl-1H-imidazol-4-yl)-N-methyl-1-(4-(trifluoromethyl)phenyl)-1H-indole-5-sulfonamide;
17)N-methyl-3-phenyl-1-(4-(trifluoromethyl)phenyl)-1H-indole-5-sulfonamide;
18) 1-(4-cyclohexylphenyl)-N-methyl-3-(1-methyl-1H-imidazol-4-yl)-1H-indole-5-sulfonamide;
19) N,N-dimethyl-3-(1-methyl-1H-imidazol-4-yl)-1-(4-(trifluoromethyl)phenyl)-1H-indole-5-sulfonamide;
20) 3-(3-fluoropyridin-2-yl)-N-methyl-1-(4-(trifluoromethyl)phenyl)-1H-indole-5-sulfonamide;
21)N-methyl-3-(1-methyl-1H-imidazol-4-yl)-1-(3-(trifluoromethyl)phenyl)-1H-indole-5-sulfonamide;
22) 1-(4-cyanophenyl)-N-methyl-3-(1-methyl-1H-imidazol-4-yl)-1H-indole-5-sulfonamide;
23) 3-(furan-3-yl)-N-methyl-1-(4-(trifluoromethyl)phenyl)-1H-indole-5-sulfonamide;
24)N-methyl-3-(5-methylfuran-2-yl)-1-phenyl-1H-indole-5-sulfonamide;
25) 3-(2,3-difluorophenyl)-N-methyl-1-(4-(trifluoromethyl)phenyl)-1H-indole-5-sulfonamide;
26)N-methyl-3-(1-methyl-1H-imidazol-4-yl)-1-phenyl-1H-indole-5-sulfonamide;
27) 1-(3-chloro-4-(trifluoromethyl)phenyl)-N-methyl-3-(1-methyl-1H-imidazol-4-yl)-1H-indole-5-sulfonamide;
28) 3-(1-cyclopropyl-1H-imidazol-4-yl)-N-methyl-1-(4-(trifluoromethyl)phenyl)-1H-indole-5-sulfonamide;
29) 3-(1H-imidazol-4-yl)-N-methyl-1-(4-(trifluoromethyl)phenyl)-1H-indole-5-sulfonamide;
30) 3-(1-(2-fluorobenzyl)-1H-imidazol-4-yl)-N-methyl-1-(4-(trifluoromethyl)phenyl)-1H-indole-5-sulfonamide;
31) 3-(furan-2-yl)-N-methyl-1-(4-(trifluoromethyl)phenyl)-1H-indole-5-sulfonamide;
32)N-methyl-3-(2-methyloxazol-5-yl)-1-(4-(trifluoromethyl)phenyl)-1H-indole-5-sulfonamide;
33)N-methyl-3-(1-methyl-1H-imidazol-4-yl)-1-(4-(trifluoromethyl)phenyl)-1H-indole-6-sulfonamide;
34)N-methyl-3-(5-(trifluoromethyl) furan-2-yl)-1-(4-(trifluoromethyl)phenyl)-1H-indole-5-sulfonamide;
35)N-methyl-3-(1-(oxetan-3-yl)-1H-imidazol-4-yl)-1-(4-(trifluoromethyl)phenyl)-1H-indole-5-sulfonamide;
36) 3-(5-chlorofuran-2-yl)-N-methyl-1-(4-(trifluoromethyl)phenyl)-1H-indole-5-sulfonamide;
37)N-methyl-3-(1-(2,2,2-trifluoroethyl)-1H-imidazol-4-yl)-1-(4-(trifluoromethyl)phenyl)-1H-indole-5-sulfonamide;
38) 3-(1-(2-methoxyethyl)-1H-imidazol-4-yl)-N-methyl-1-(4-(trifluoromethyl)phenyl)-1H-indole-5-sulfonamide;
39)N-methyl-3-(1-methyl-1H-imidazol-4-yl)-1-(p-tolyl)-1H-indole-5-sulfonamide;
40) 1-(4-(t-butyl)phenyl)-N-methyl-3-(1-methyl-1H-imidazol-4-yl)-1H-indole-5-sulfonamide;
41)N-methyl-3-(oxazol-5-yl)-1-(4-(trifluoromethyl)phenyl)-1H-indole-5-sulfonamide;
42) 3-(1-cyclopropyl-1H-imidazol-4-yl)-1-(2-fluoro-4-(trifluoromethyl)phenyl)-N-methyl-1H-indole-5-sulfonamide;
43) 3-(1-cyclopropyl-1H-imidazol-4-yl)-N-methyl-1-(4-(trifluoromethyl)phenyl)-1H-pyrrolo[2,3-b]pyridin-5-sulfonamide;
44) 3-(1-cyclopropyl-1H-imidazol-4-yl)-N-methyl-1-(4-(trifluoromethyl)phenyl)-1H-indazol-5-sulfonamide;
45) 3-(6,7-dihydro-5H-pyrrolo[1,2-a]imidazol-2-yl)-N-methyl-1-(4-(trifluoromethyl)phenyl)-1H-indole-5-sulfonamide;
46) 3-(3-fluoroazetidin-1-yl)-N-methyl-1-(4-(trifluoromethyl)phenyl)-1H-indole-5-sulfonamide;
47) N,N-dimethyl-2-(4-(5-(N-methylsulfamoyl)-1-(4-(trifluoromethyl)phenyl)-1H-indol-3-yl)-1H-imidazole-1-yl) acetamide; and
48)N-methyl-3-(1-(2-(methylsulfonyl)ethyl)-1H-imidazol-4-yl)-1-(4-(trifluoromethyl)phenyl)-1H-indole-5-sulfonamide.

According to an embodiment of the present disclosure, provided is a pharmaceutical composition for treating or preventing dysregulation of the Hippo signaling pathway, specifically related diseases caused by TEAD activation, the pharmaceutical composition including, as an active ingredient, the compound selected from the compounds of Formula 1, the enantiomers, diastereomers, solvates and hydrates thereof, and the pharmaceutically acceptable salts thereof.

In an embodiment, the composition may exhibit inhibitory activity against Yes associated protein (YAP)-transcriptional enhancer associate domain (TEAD) binding.

In an embodiment, the composition may be for treating treatable cancer or tumor by exhibiting inhibitory activity against YAP-TEAD binding.

In an embodiment, the pharmaceutical composition may include, in a therapeutically effective amount, the compound selected from the compounds of Formula 1, the enantiomers, diastereomers, solvates and hydrates, and the pharmaceutically acceptable salt thereof.

As used herein, the term “therapeutically effective amount” refers to an amount of a compound of the present disclosure that treats or prevents a particular disease, condition or disorder, attenuates, ameliorates or eliminates one or more symptoms of a particular disease, condition or disorder, or prevents or delays the onset of one or more symptoms of a particular disease, condition or disorder.

A physician of ordinary skill in the relevant art may readily determine and prescribe an effective required dose of a pharmaceutical composition. For example, the pharmaceutical composition may include, 0.0001 mg to 10 g of the compound, but is not limited thereto.

In an embodiment, the pharmaceutical composition may further include a pharmaceutically acceptable additive in addition to the active ingredient. The additive may be, for example, diluents, disintegrating agents, binders, lubricant, surfactants, suspending agents, or emulsifiers, etc., but is not limited thereto.

The pharmaceutical compositions of the present disclosure may be formulated according to usual methods and may be prepared in various oral dosage forms, such as tablets, pills, powders, capsules, syrups, emulsions, microemulsions, or parenteral dosage forms, such as intramuscular, intravenous, or subcutaneous administration.

Another embodiment of the present disclosure provides a treating method of administering the pharmaceutical composition to a subject suffering from a dysregulation of the Hippo signaling pathway, specifically a related disease caused by TEAD activation, the pharmaceutical composition including, as an active ingredient, the compound selected from the compounds of Formula 1, the enantiomers, diastereomers, solvates and hydrates, and the pharmaceutically acceptable salt thereof.

As used herein, the term “treating” or “curing” refers to inhibiting a disease, for example, inhibiting a disease, condition, or disorder in a subject experiencing or exhibiting the pathology or symptoms of the disease, condition, or disorder, for example, preventing or reversing further development of the pathology and/or symptoms, or ameliorating a disease, for example, reducing disease severity.

As used herein, the term “preventing” or “prophylactic” refers to preventing a disease, for example, preventing a disease, condition, or disorder in an individual who may have a predisposition to the disease, condition, or disorder but has not yet experienced or exhibited the pathology or symptoms of the disease.

As used herein, the term “subject” or “individual” may be a vertebrate such as a mammal, fish, bird, reptile, or amphibian. For example, a subject may be a human, non-human primate, horse, pig, rabbit, dog, sheep, goat, cow, cat, guinea pig, or rodent.

As used herein, the terms “administering” and “administration” refer to any method of providing a disclosed composition to a subject.

The dosage, number of doses, or method of administration of a compound or pharmaceutical composition according to an embodiment may vary depending on the subject being treated, the severity of the disease or condition, the rate of administration, and the judgment of the prescribing physician. For example, a typical dosage for a person weighing 70 kg may be administered in an amount from 0.0001 mg to 10 g per day, for example 1 mg to 1 g per day. The number of doses may be administered once to several times, for example 1 to 4 times, or on an on/off schedule, and the method of administration may be by an oral or parenteral route. For example, a compound or pharmaceutical composition according to an embodiment may be administered by an oral or parenteral route in an amount ranging from 0.1 to 100 mg/kg (body weight).

The physician may start at a lower level than necessary to achieve the target therapeutic effect and gradually increase the dose of the compound or pharmaceutical composition of the present disclosure administered to the subject until the intended effect is achieved.

Another embodiment of the present disclosure provides a kit including as an active ingredient a compound selected from compounds of Formula 1, pharmaceutically acceptable salts, enantiomers, diastereomers, hydrates, and solvates thereof.

In an embodiment, the therapeutic agent may be a drug for treating a related disease caused by dysregulation of the Hippo signaling pathway, specifically TEAD activation, for example a drug for treating cancer. For example, the therapeutic agent may be a chemotherapeutic agent for the treatment of cancer.

In an embodiment, the compounds, compositions, and kits of the present disclosure may be administered alone or simultaneously with at least one other therapeutic agent, separately or sequentially.

In the context of the present disclosure, the singular form of a word may include the plural and vice versa, unless the context clearly indicates otherwise.

Figures given herein shall be deemed to include the meaning of “about” even if not expressly stated. As used herein, the term “about” refers to within 5% of a certain value or range, preferably within 1% to 2%.

As used herein, the numerical range indicated using the term “to” refer to a range that includes the numerical values written before and after the term “to” as the lower limit and upper limit, respectively.

As used herein, the terms “has,” “may have,” “includes,” or “may include” refer to the presence of a particular feature (for example, a number, or a component such as an ingredient) and do not exclude the presence of additional features.

The contents of all publications incorporated by reference herein are hereby incorporated by reference in their entirety.

Hereinafter, a method of preparing a compound of formula 1 will be described in detail.

The compound of formula 1 according to the present disclosure may be prepared according to the synthetic method shown in Reaction 1.

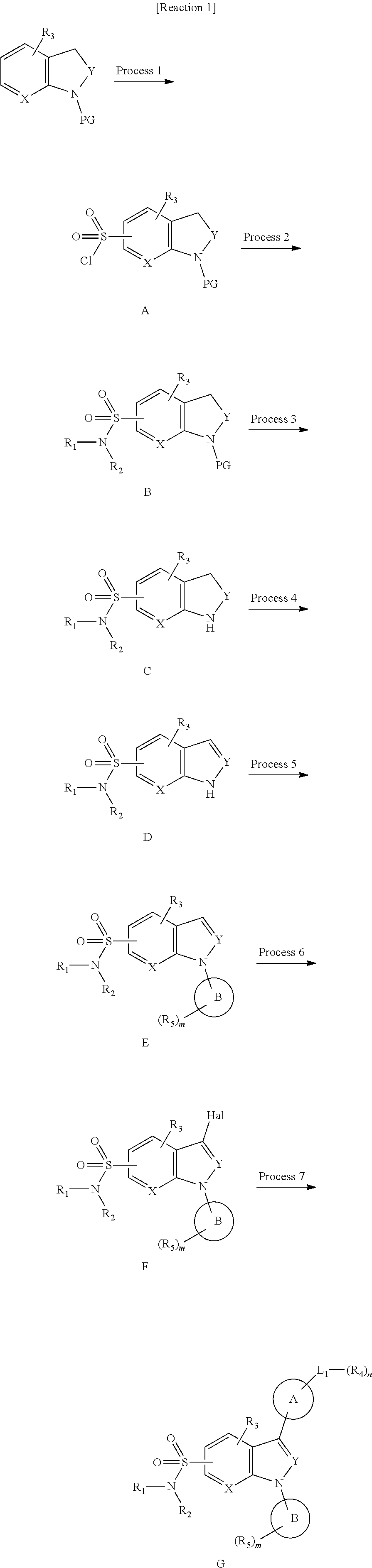

### [Process-1]

A starting material (for example, PG-indoline; 1 equivalent, standard equivalent) is slowly added to chlorosulfonic acid (7.5 equivalents) under cooling conditions at 0° C. to 5° C. After the reaction temperature was raised to room temperature, the reaction mixture was heated to react at 70° C. After confirming the completion of the reaction, the reaction solution is slowly added dropwise to water cooled to 0° C. to 5° C., and collect the formed solid by filtration to obtain the target compound A.

### [Process-2]

Triethylamine (2 equivalents) is added to the reaction solution of A (1 equivalent, standard equivalent) prepared in [Process-1] above dissolved in dichloromethane. The corresponding amine solution (2 equivalents) is added dropwise thereto and the reaction solution is stirred under reflux. After confirming the completion of the reaction, the reaction mixture is cooled to room temperature, and the formed solid is collected by filtration to obtain the target compound B.

### [Process-3]

Compound B (1 equivalent, standard equivalent) obtained in [Process-2] above is dissolved in methanol, and concentrated HCl (4 equivalents) is added thereto. The reaction solution is stirred at room temperature overnight, heated to 80° C., and stirred for additional 2 hours. After confirming the completion of the reaction, the internal temperature is lowered to room temperature, water is added, and sodium hydroxide aqueous solution is slowly added dropwise to adjust the pH to 3 to 4. The formed solid is collected by filtration to obtain the target compound C.

### [Process-4]

Compound C (1 equivalent, standard equivalent) obtained in [Process-3] above is dissolved in dichloromethane, and 2,3-dichloro-5,6-dicyano-1,4-benzoquinone (DDQ, 1 equivalent) is added thereto. Then, the reaction solution is stirred at room temperature overnight. After confirming the completion of the reaction, the reaction solution is filtered through CELITE and washed with dichloromethane. The residue obtained after concentration under reduced pressure is purified by MPLC to obtain the target compound D.

### [Process-5]

Compound D (1 equivalent, standard equivalent) obtained in the above [Process-4] is dissolved in dimethylformamide, and the corresponding halogen-substituted B derivative (1.5 equivalents) and potassium carbonate (2.5 equivalents) are added thereto, followed by addition of copper iodide (0.2 equivalent), and the reaction solution is stirred at room temperature overnight. After confirming the completion of the reaction, the reaction mixture is cooled to room temperature and water is added thereto, followed by extraction with ethyl acetate. The organic layer thus obtained is dried over anhydrous sodium sulfate, filtered under reduced pressure, and the filtered organic layer is concentrated under reduced pressure. The obtained residue is purified by MPLC to obtain the target compound E.

### [Process-6]

Compound E (1 equivalent, standard equivalent) obtained in [Process-5] above is dissolved in dimethylformamide, and N-bromosuccinimide (1 equivalent) is slowly added dropwise thereto. Then, the reaction solution was stirred at room temperature. After confirming the completion of the reaction, water was added and extraction is performed thereon with ethyl acetate. The organic layer is dried over anhydrous sodium sulfate, filtered under reduced pressure, and the filtered organic layer is concentrated under reduced pressure. The obtained residue is purified by MPLC to obtain the target compound F.

### [Process-7]

Compound F (1 equivalent, standard equivalent) obtained in [Process-6] above is subjected to a Stille coupling reaction or Suzuki coupling reaction with the corresponding A-stannane derivative and A-borane derivative (2 equivalents). After completion of the reaction, the organic layer is washed with water, dried over anhydrous sodium sulfate, filtered under reduced pressure, and the filtered organic layer is concentrated under reduced pressure. The obtained residue is purified by MPLC to obtain the target compound G.

In Reaction 1, R₁, R₂, R₃, R₄, R₅, X, Y, m, n, L₁, \textcircled{A}, and \textcircled{B}, respectively, are as defined in Formula 1 above, but not limited thereto and may be changed within the scope understood by those skilled in the art.

The compound of Formula 1 according to an embodiment of the present disclosure may be prepared according to, but not limited to, the method shown in Reaction 1 above. A person with ordinary knowledge in the field of organic compounds may appropriately adjust the specific reaction pathway, reaction conditions, reaction amount, etc.

Hereinafter, the present disclosure will be further described in more detail through the following embodiments and experimental examples. However, these embodiments and examples are intended only to illustrate the present disclosure and are not intended to limit the scope of the present disclosure in any way.

### Example 1: N-methyl-3-(3-methylpyrazin-2-yl)-1-(4-(trifluoromethyl)phenyl)-1H-indole-5-sulfonamide

### [Process-1] Preparation of 1-acetylindoline-5-sulfonyl Chloride

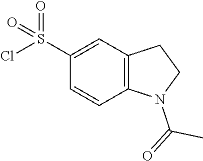 [SMILES: CC(=O)N1CCc2cc(S(=O)(=O)Cl)ccc21]

1-indoline-1-yl ethanone (30 g, 124.07 mmol) was added to chlorosulfonic acid (62 mL, 930.5 mmol) in small portions over 15 minutes under cooling to 0° C. to 5° C. After the temperature was raised to room temperature, the reaction mixture was heated to 70° C. for 90 minutes. After confirming the completion of the reaction, the reaction solution was slowly added dropwise to water cooled to 0° C. to 5° C. The formed solid was collected by filtration, washed several times with water, and dried at 50° C. to obtain 27 g (55% yield) of the title compound.

¹H-NMR (300 MHz, DMSO-d₆): δ 7.96-7.93 (m, 1H), 7.43-7.38 (m, 2H), 4.10 (t, J=8.5 Hz, 2H), 3.13 (t, J=8.5 Hz, 2H), 2.16 (s, 3H).

### [Process-2] Preparation of 1-acetyl-N-methylindoline-5-sulfonamide

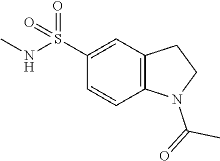 [SMILES: CNS(=O)(=O)c1ccc2c(c1)CCN2C(C)=O]

1-acetylindoline-5-sulfonyl chloride (20 g, 77 mmol) obtained in [Process-1] above was dissolved in in 60 mL of dichloromethane, and triethylamine (21.5 mL, 154 mmol) was added thereto. To this mixture, a 2M methylamine tetrahydrofuran solution (77 mL, 154 mmol) was added dropwise, and the reaction solution was refluxed and stirred. After confirming the completion of the reaction, the mixture was cooled to room temperature. The formed solid was collected by filtration, washed several times with water, and dried at 50° C. to obtain 18.8 g (96% yield) of the title compound.

¹H-NMR (300 MHz, DMSO-d₆): δ 8.16-8.14 (m, 1H), 7.60-7.57 (m, 2H), 7.30 (s, 1H), 4.16 (t, J=8.6 Hz, 2H), 3.21 (t, J=8.5 Hz, 2H), 2.39 (s, 3H), 2.19 (s, 3H).

### [Process-3] Preparation of N-methyl-1H-indoline-5-sulfonamide

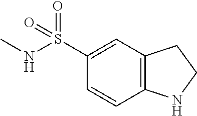

1-acetyl-N-methylindoline-5-sulfonamide (6.2 g, 24.2 mmol) obtained in [Process-2] above was dissolved in 72 mL of methanol, and concentrated HCl (8.1 mL, 97.5 mmol) was added thereto. The reaction solution was stirred at room temperature overnight and then stirred at 80° C. for 2 hours. After confirming the completion of the reaction, the internal temperature was lowered to room temperature, 100 ml of water was added, and then 1 N sodium hydroxide aqueous solution was slowly added dropwise to adjust to pH 3 to 4. The formed solid was collected by filtration, washed several times with water, and dried at 50° C. to obtain 3.4 g (66% yield) of the title compound.

¹H-NMR (300 MHz, CDCl₃): δ 7.56-7.53 (m, 2H), 6.66-6.61 (m, 1H), 4.17 (brs, 1H), 3.71 (t, J=8.6 Hz, 2H), 3.11 (t, J=8.5 Hz, 2H), 2.64 (d, J=5.5 Hz, 3H), 1.59 (brs, 1H).

### [Process-4] Preparation of N-methyl-1H-indole-5-sulfonamide

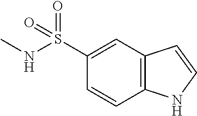

N-methyl-1H-indoline-5-sulfonamide (3.3 g, 15.5 mmol) prepared in [Process-3] above was dissolved in 30 mL of dichloromethane, and 2,3-dichloro-5,6-dicyano-1,4-benzoquinone (DDQ, 3.5 g, 15.5 mmol) was added thereto. Then, the reaction solution was stirred at room temperature overnight. After completion of the reaction, the reaction solution was filtered through CELITE and washed with dichloromethane. The residue obtained after concentration under reduced pressure was purified by MPLC (ethyl acetate:hexane=1:3 (v/v)) to obtain 1.9 g (58% yield) of the title compound.

¹H-NMR (300 MHz, CDCl₃): δ 8.52 (s, 1H), 8.26-8.25 (m, 1H), 7.77-7.68 (m, 1H), 7.53-7.51 (m, 1H), 7.39-7.37 (m, 1H), 6.72-6.71 (m, 1H), 4.24 (brs, 1H), 2.67 (d, J=5.5 Hz, 3H).

### [Process-5] Preparation of N-methyl-1-(4-(trifluoromethyl)phenyl)-1H-indole-5-sulfonamide

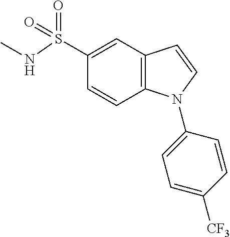 [SMILES: CNS(=O)(=O)c1ccc2c(ccn2-c2ccc(C(F)(F)F)cc2)c1]

N-methyl-1H-indole-5-sulfonamide (1.9 g, 9.04 mmol) obtained in [Process-4] above was dissolved in 20 mL of dimethylformamide, and 4-bromobenzotrifluoride (3.08 g, 13.6 mmol) and potassium carbonate (7.36 g, 22.6 mmol) were added thereto, followed by addition of copper iodide (344 mg, 1.8 mmol). The reaction solution was stirred at 130° C. overnight. After completion of the reaction, the mixture was cooled to room temperature, 100 ml of water was added thereto, and extraction was performed thereon three times with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:1 (v/v)) to obtain 1.2 g (37% yield) of the title compound.

¹H-NMR (300 MHz, CDCl₃): δ 8.31 (s, 1H), 7.87-7.84 (m, 2H), 7.76-7.73 (m, 1H), 7.67-7.51 (m, 3H), 7.49 (s, 1H), 6.88-6.87 (m, 1H), 4.35-4.11 (m, 1H), 2.70 (d, J=5.5 Hz, 3H).

### [Process-6] Preparation of 3-bromo-N-methyl-1-(4-(trifluoromethyl)phenyl)-1H-indole-5-sulfonamide

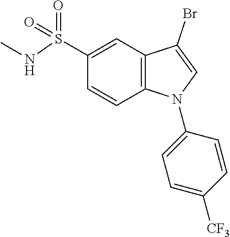 [SMILES: CNS(=O)(=O)c1ccc2c(c1)c(Br)cn2-c1ccc(C(F)(F)F)cc1]

N-methyl-1-(4-(trifluoromethyl)phenyl)-1H-indole-5-sulfonamide (520 mg, 1.47 mmol) obtained in [Process-5] above was dissolved in 30 mL of dimethylformamide, and N-bromosuccinimide (261 mg, 1.47 mmol) was slowly added dropwise thereof. The reaction solution was stirred at room temperature for 3 hours to complete the reaction, 100 ml of water was added thereto, and extraction was performed thereon three times with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:1 (v/v)) to obtain 420 mg (66% yield) of the title compound.

¹H-NMR (300 MHz, CDCl₃): δ 8.26-8.25 (m, 1H), 7.89-7.79 (m, 3H), 7.67-7.62 (m, 3H), 7.55 (s, 1H), 4.41-4.26 (m, 1H), 2.72 (s, 3H).

### [Process-7] Preparation of N-methyl-3-(3-methylpyrazin-2-yl)-1-(4-(trifluoromethyl)phenyl)-1H-indole-5-sulfonamide

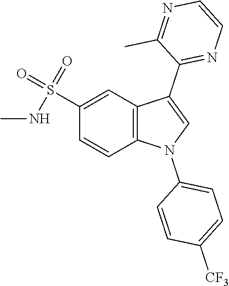 [SMILES: CNS(=O)(=O)c1ccc2c(c1)c(-c1nccnc1C)cn2-c1ccc(C(F)(F)F)cc1]

3-bromo-N-methyl-1-(4-(trifluoromethyl)phenyl)-1H-indole-5-sulfonamide (50 mg, 0.12 mmol) obtained in [Process-6] above was dissolved in 2 mL of N,N-dimethylacetamide and copper chloride (3 mg, 0.02 mmol), and cesium fluoride (53 mg, 0.35 mmol), [1,1′-bis(diphenylphosphino)bisferrocenyl]palladium dichloride ([Pd(dppf)Cl₂]), (16 mg, 0.02 mmol), and tributyl-(3-methylpyrazin-2-yl)stannane (88 mg, 0.23 mmol) were added thereto respectively. The reaction solution was stirred at 120° C. overnight to complete the reaction, 20 ml of water was added thereto, and extraction was performed thereon three times with ethyl acetate. The organic layer thus obtained was dried over anhydrous sodium sulfate, filtered under reduced pressure, and the filtered organic layer was concentrated under reduced pressure. The obtained residue was purified by MPLC (dichloromethane:methanol=20:1 (v/v)) to obtain 18 mg (6% yield) of the title compound.

¹H-NMR (300 MHz, CDCl₃): δ 8.77 (s, 1H), 8.59 (s, 1H), 8.45 (s, 1H), 7.83-7.80 (m, 2H), 7.68-7.65 (m, 3H), 4.88-4.87 (m, 1H), 2.80 (s, 3H), 2.67 (s, 3H). MS (ESI⁺, m/z): 447.1 [M+H]⁺

### Example 2: N-methyl-3-(1-methyl-1H-imidazol-4-yl)-1-(4-(trifluoromethyl)phenyl)-1H-indole-5-sulfonamide

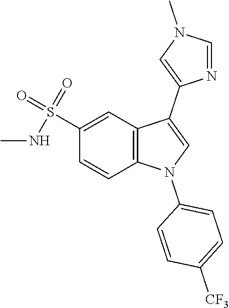 [SMILES: CNS(=O)(=O)c1ccc2c(c1)c(-c1cn(C)cn1)cn2-c1ccc(C(F)(F)F)cc1]

The procedure of Example 1 was repeated except for the use of tributyl-(1-methylimidazol-4-yl)stannane (171 mg, 0.46 mmol) instead of tributyl-(3-methylpyrazin-2-yl)stannane in [Process-7] of Example 1 above, to obtain 30 mg (30% yield) of the title compound.

¹H-NMR (300 MHz, CDCl₃): δ 8.56 (s, 1H), 7.94 (s, 1H), 7.77-7.76 (m, 6H), 7.54 (s, 1H), 7.37 (s, 1H), 4.83-4.81 (m, 1H), 3.81 (s, 3H), 2.69 (d, J=5.4 Hz, 3H).

MS (ESI⁺, m/z): 435.1 [M+H]⁺

### Example 3: N-methyl-3-(1-methyl-1H-pyrazol-3-yl)-1-(4-(trifluoromethyl)phenyl)-1H-indole-5-sulfonamide

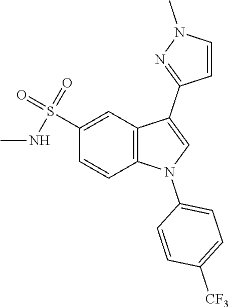 [SMILES: CNS(=O)(=O)c1ccc2c(c1)c(-c1ccn(C)n1)cn2-c1ccc(C(F)(F)F)cc1]

3-bromo-N-methyl-1-(4-(trifluoromethyl)phenyl)-1H-indole-5-sulfonamide (50 mg, 0.11 mmol) obtained in [Process-6] of Example 1 above was dissolved in 2.5 mL of 1,4-dioxane:water (1:4, v/v), and 1-methyl-3-(4,4,5,5-tetramethyl-1,3,2-dioxaborolan-2-yl) pyrazole (27 mg, 0.12 mmol), [1,1′-bis(diphenylphosphino)bisferrocenyl]palladium dichloride ([Pd(dppf)Cl₂]) (9 mg, 0.01 mmol) and sodium carbonate (24 mg, 0.23 mmol) were added thereto respectively. After the reaction solution was stirred at 100° C. overnight to complete the reaction, the reaction solution was cooled to room temperature, 10 ml of water was added thereto, and extraction was performed thereon three times with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:1 (v/v)) to obtain 15 mg (30% yield) of the title compound.

¹H-NMR (300 MHz, CD₃OD): δ 8.75 (s, 1H), 8.05 (s, 1H), 7.95-7.92 (m, 2H), 7.87-7.84 (m, 2H), 7.80-7.73 (m, 2H), 7.67-7.66 (m, 1H), 6.67 (s, 1H), 4.00 (s, 3H), 2.54 (s, 3H).

MS (ESI⁺, m/z): 435.2 [M+H]⁺

### Example 4: N-methyl-3-(1-methyl-1H-pyrazol-4-yl)-1-(4-(trifluoromethyl)phenyl)-1H-indole-5-sulfonamide

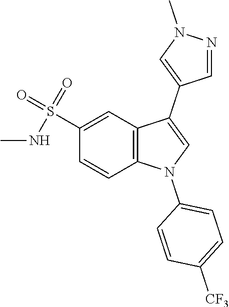 [SMILES: CNS(=O)(=O)c1ccc2c(c1)c(-c1cnn(C)c1)cn2-c1ccc(C(F)(F)F)cc1]

The procedure of Example 3 was repeated except for the use of 1-methyl-4-(4,4,5,5-tetramethyl-1,3,2-dioxaboran-2-yl) pyrazole (27 mg, 0.13 mmol) instead of 1-methyl-3-(4,4,5,5-tetramethyl-1,3,2-dioxaborolan-2-yl) pyrazole in Example 3 above, to obtain 11 mg (21% yield) of the title compound.

¹H-NMR (300 MHz, CDCl₃): δ 8.35 (s, 1H), 7.86-7.83 (m, 2H), 7.78-7.74 (m, 3H), 7.67-7.60 (m, 3H), 7.55 (s, 1H), 4.03 (s, 3H), 2.67 (d, J=5.5 Hz, 3H).

MS (ESI⁺, m/z): 435.2 [M+H]⁺

### Example 5: N-methyl-3-(1-methyl-1H-imidazol-4-yl)-1-(4-(trifluoromethoxy)phenyl)-1H-indole-5-sulfonamide

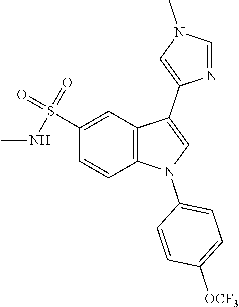 [SMILES: CNS(=O)(=O)c1ccc2c(c1)c(-c1cn(C)cn1)cn2-c1ccc(OC(F)(F)F)cc1]

The procedure of Example 1 was repeated, except for the use of 1-bromo-4-(trifluoromethoxy)benzene (443 mg, 1.78 mmol) instead of 4-bromobenzotrifluoride in [Process-5], the use of tributyl-(1-methylimidazol-4-yl)stannane (223 mg, 0.60 mmol) instead of tributyl-(3-methylpyrazin-2-yl)stannane in [Process-7], and the use of ethyl acetate:hexane=2:1 (v/v) instead of dichloromethane:methanol=20:1 (v/v) during purification, of Example 1 above, to obtain 20 mg (14% yield) of the title compound.

¹H-NMR (300 MHz, CDCl₃): δ 8.50 (d, J=1.6 Hz, 1H), 7.86 (s, 1H), 7.74-7.70 (m, 1H), 7.61-7.53 (m, 4H), 7.43-7.40 (m, 2H), 7.31 (s, 1H), 4.31-4.29 (m, 1H), 3.80 (s, 3H), 2.66 (d, J=5.5 Hz, 3H).

MS (ESI⁺, m/z): 451.2 [M+H]⁺

### Example 6:3-(1-isopropyl-1H-imidazol-4-yl)-N-methyl-1-(4-(trifluoromethyl)phenyl)-1H-indole-5-sulfonamide

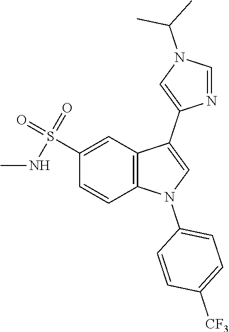 [SMILES: CNS(=O)(=O)c1ccc2c(c1)c(-c1cn(C(C)C)cn1)cn2-c1ccc(C(F)(F)F)cc1]

The procedure of Example 1 was repeated, except for the use of tributyl-(1-isopropylimidazol-4-yl)stannane (95 mg, 0.23 mmol) instead of tributyl-(3-methylpyrazin-2-yl)stannane in [Process-7] and the use of ethyl acetate:hexane=2:1 (v/v) instead of dichloromethane:methanol=20:1 (v/v) during purification, of Example 1 above, to obtain 39 mg (71% yield) of the title compound.

¹H-NMR (300 MHz, CDCl₃): δ 8.14 (d, J=1.1 Hz, 1H), 7.89-7.78 (m, 4H), 7.70-7.67 (m, 3H), 7.50 (s, 1H), 7.18 (s, 1H), 4.39-4.29 (m, 2H), 2.66 (d, J=5.4 Hz, 3H), 1.48 (d, J=6.8 Hz, 6H).

MS (ESI⁺, m/z): 463.2 [M+H]⁺

### Example 7: N-ethyl-3-(1-methyl-1H-imidazol-4-yl)-1-(4-(trifluoromethyl)phenyl)-1H-indole-5-sulfonamide

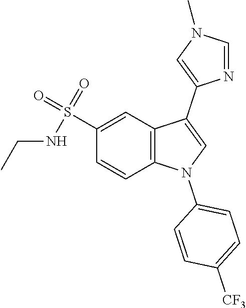 [SMILES: CCNS(=O)(=O)c1ccc2c(c1)c(-c1cn(C)cn1)cn2-c1ccc(C(F)(F)F)cc1]

The procedure of Example 1 was repeated except for using 2 M ethylamine tetrahydrofuran solution (27 mL) instead of 2 M methylamine tetrahydrofuran solution in [Process-2] and tributyl-(1-methylimidazol-4-yl)stannane (171 mg, 0.46 mmol) instead of tributyl-(3-methylpyrazin-2-yl)stannane in [Process-7], of Example 1 above, to obtain 17 mg (17% yield) of the title compound.

¹H-NMR (300 MHz, DMSO-d₆): δ 8.74 (s, 1H), 8.16 (s, 1H), 8.01-7.94 (m, 4H), 7.84-7.81 (m, 1H), 7.74 (s, 1H), 7.70-7.67 (m, 1H), 7.56 (s, 1H), 7.48-7.44 (m, 1H), 3.76 (s, 3H), 2.80-2.76 (m, 2H), 0.98 (t, J=7.3 Hz, 3H).

MS (ESI⁺, m/z): 449.1 [M+H]⁺

### Example 8: N-methyl-3-(pyridin-2-yl)-1-(4-(trifluoromethyl)phenyl)-1H-indole-5-sulfonamide

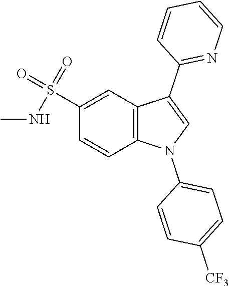 [SMILES: CNS(=O)(=O)c1ccc2c(c1)c(-c1ccccn1)cn2-c1ccc(C(F)(F)F)cc1]

The procedure of Example 1 was repeated, except for the use of tributyl-(2-pyridinyl)stannane (85 mg, 0.23 mmol) instead of tributyl-(3-methylpyrazin-2-yl)stannane in [Process-7] and the use of ethyl acetate:hexane=1:1 (v/v) instead of dichloromethane:methanol=20:1 (v/v) during purification, of Example 1 above, to obtain 7 mg (14% yield) of the title compound.

¹H-NMR (300 MHz, DMSO-de): δ 9.15 (s, 1H), 8.73-8.71 (m, 2H), 8.04-8.01 (m, 5H), 7.98-7.83 (m, 2H), 7.72-7.68 (m, 1H), 7.40-7.39 (m, 1H), 7.30-7.26 (m, 1H), 2.42 (d, J=4.8 Hz, 3H).

MS (ESI⁺, m/z): 432.2 [M+H]⁺

### Example 9: N-methyl-3-(5-methylthiophen-2-yl)-1-(4-(trifluoromethyl)phenyl)-1H-indole-5-sulfonamide

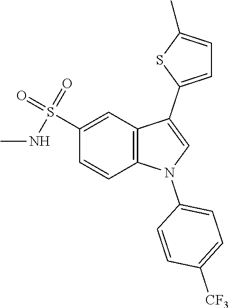 [SMILES: CNS(=O)(=O)c1ccc2c(c1)c(-c1ccc(C)s1)cn2-c1ccc(C(F)(F)F)cc1]

The procedure of Example 3 was repeated except for the use of 4,4,5,5-tetramethyl-2-(5-methyl-2-thienyl)-1,3,2-dioxaboran (29 mg, 0.12 mmol) instead of 1-methyl-3-(4,4,5,5-tetramethyl-1,3,2-dioxaboran-2-yl) pyrazole in Example 3 above, to obtain 26 mg (52% yield) of the title compound.

¹H-NMR (300 MHz, CDCl₃): δ 8.54 (s, 1H), 7.86-7.84 (m, 2H), 7.78-7.75 (m, 1H), 7.67-7.62 (m, 3H), 7.59 (s, 1H), 7.15-7.14 (m, 1H), 6.82-6.81 (m, 1H), 4.30-4.25 (m, 1H), 2.69-2.67 (m, 3H), 2.56-2.55 (m, 3H).

MS (ESI⁺, m/z): 451.2 [M+H]⁺

### Example 10: N-methyl-3-(5-methylfuran-2-yl)-1-(4-(trifluoromethyl)phenyl)-1H-indole-5-sulfonamide

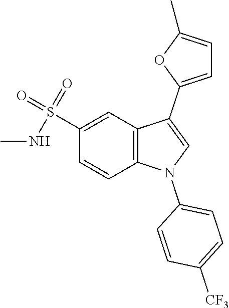 [SMILES: CNS(=O)(=O)c1ccc2c(c1)c(-c1ccc(C)o1)cn2-c1ccc(C(F)(F)F)cc1]

The procedure of Example 3 was repeated except for the use of 4,4,5,5-tetramethyl-2-(5-methyl-2-furyl)-1,3,2-dioxaboran (27 mg, 0.12 mmol) instead of 1-methyl-3-(4,4,5,5-tetramethyl-1,3,2-dioxaboran-2-yl) pyrazole in Example 3 above, to obtain 29 mg (50% yield) of the title compound.

¹H-NMR (300 MHz, CDCl₃): δ 8.54-8.53 (m, 1H), 7.86-7.83 (m, 2H), 7.78-7.62 (m, 5H), 6.60-6.59 (m, 1H), 6.14-6.12 (m, 1H), 4.31-4.26 (m, 1H), 2.70-2.68 (m, 3H), 2.41 (s, 3H).

MS (ESI⁺, m/z): 435.20 [M+H]⁺

### Example 11:3-(1-ethyl-1H-imidazol-4-yl)-N-methyl-1-(4-(trifluoromethyl)phenyl)-1H-indole-5-sulfonamide

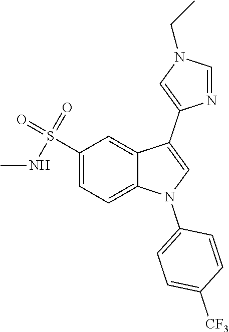 [SMILES: CCn1cnc(-c2cn(-c3ccc(C(F)(F)F)cc3)c3ccc(S(=O)(=O)NC)cc23)c1]

The procedure of Example 1 was repeated except for the use of tributyl-(1-ethylimidazol-4-yl)stannane (73 mg, 0.18 mmol) instead of tributyl-(3-methylpyrazin-2-yl)stannane in [Process-7] of Example 1 above, to obtain 9 mg (17% yield) of the title compound.

¹H-NMR (300 MHz, CDCl₃): δ 8.52 (s, 1H), 7.92 (s, 1H), 7.84 (d, J=8.9 Hz, 2H), 7.73-7.66 (m, 4H), 7.60-7.59 (m, 2H), 7.35 (s, 1H), 4.12-4.09 (m, 2H), 2.67 (d, J=5.4 Hz, 3H), 1.25 (s, 3H).

MS (ESI⁺, m/z): 449.2 [M+H]⁺

### Example 12: N-methyl-3-(1-methyl-1H-imidazol-4-yl)-1-(5-(trifluoromethyl)pyridin-2-yl)-1H-indole-5-sulfonamide

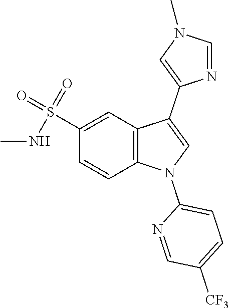 [SMILES: CNS(=O)(=O)c1ccc2c(c1)c(-c1cn(C)cn1)cn2-c1ccc(C(F)(F)F)cn1]

The procedure of Example 5 was repeated except for the use of 2-bromo-5-(trifluoromethyl)pyridine (427 mg, 1.85 mmol) instead of 1-bromo-4-(trifluoromethoxy)benzene in Example 5 above, to obtain 21 mg (34% yield) of the title compound.

¹H-NMR (300 MHz, CD₃OD): δ 8.90 (s, 1H), 8.78-8.75 (m, 1H), 8.44 (s, 1H), 8.34 (s, 1H), 8.29-8.25 (m, 1H), 7.91-7.88 (m, 1H), 7.83-7.79 (m, 2H), 7.58 (s, 1H) 3.85 (s, 3H), 2.55 (s, 3H).

MS (ESI⁺, m/z): 436.2 [M+H]⁺

### Example 13:3-(2-fluorophenyl)-N-methyl-1-(4-(trifluoromethyl)phenyl)-1H-indole-5-sulfonamide

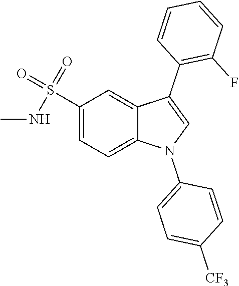 [SMILES: CNS(=O)(=O)c1ccc2c(c1)c(-c1ccccc1F)cn2-c1ccc(C(F)(F)F)cc1]

The procedure of Example 3 was repeated except for the use of (2-fluorophenyl) boronic acid (21 mg, 0.15 mmol) instead of 1-methyl-3-(4,4,5,5-tetramethyl-1,3,2-dioxaborolan-2-yl) pyrazole in Example 3 above, to obtain 5.5 mg (10% yield) of the title compound.

¹H-NMR (300 MHz, CDCl₃): δ 8.40 (s, 1H), 7.87 (d, J=8.4 Hz, 2H), 7.99 (d, J=1.8 Hz, 1H), 7.77-7.66 (m, 5H), 7.36-7.28 (m, 3H), 4.26-4.24 (m, 1H), 2.67 (d, J=5.5 Hz, 3H).

MS (ESI⁺, m/z): 449.2 [M+H]⁺

### Example 14:1-(4-chlorophenyl)-N-methyl-3-(1-methyl-1H-imidazol-4-yl)-1H-indole-5-sulfonamide

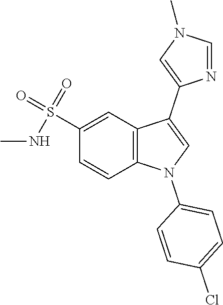 [SMILES: CNS(=O)(=O)c1ccc2c(c1)c(-c1cn(C)cn1)cn2-c1ccc(Cl)cc1]

The procedure of Example 5 was repeated except for the use of 1-bromo-4-chloro-benzene (344 mg, 1.78 mmol) instead of 1-bromo-4-(trifluoromethoxy)benzene in Example 5 above, to obtain 10 mg (15% yield) of the title compound.

¹H-NMR (300 MHz, CDCl₃): δ 8.52 (d, J=1.5 Hz, 1H), 7.87 (s, 1H), 7.75-7.72 (m, 1H), 7.61-7.47 (m, 6H), 7.33 (d, J=7.0 Hz, 1H), 4.44-4.39 (m, 1H), 3.82 (s, 3H), 2.69 (d, J=5.4 Hz, 3H).

MS (ESI⁺, m/z): 401.1 [M+H]⁺

### Example 15: N-methyl-3-(pyridin-4-yl)-1-(4-(trifluoromethyl)phenyl)-1H-indole-5-sulfonamide

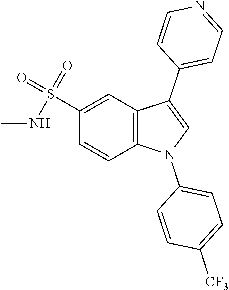 [SMILES: CNS(=O)(=O)c1ccc2c(c1)c(-c1ccncc1)cn2-c1ccc(C(F)(F)F)cc1]

The procedure of Example 1 was repeated except for the use of tributyl-(4-pyridyl)stannane (73 mg, 0.19 mmol) instead of tributyl-(3-methylpyrazin-2-yl)stannane in [Process-7] of Example 1 above, to obtain 5 mg (9% yield) of the title compound.

¹H-NMR (300 MHz, CDCl₃): δ 8.54 (s, 1H), 8.32 (s, 1H), 7.99-7.89 (m, 8H), 7.87-7.83 (m, 2H), 2.54 (s, 3H).

MS (ESI⁺, m/z): 432.2 [M+H]⁺

### Example 16: 3-(1-cyclobutyl-1H-imidazol-4-yl)-N-methyl-1-(4-trifluoromethyl)phenyl)-1H-indole-5-sulfonamide

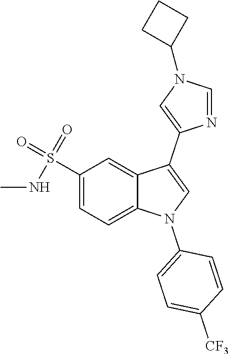 [SMILES: CNS(=O)(=O)c1ccc2c(c1)c(-c1cn(C3CCC3)cn1)cn2-c1ccc(C(F)(F)F)cc1]

The procedure of Example 1 was repeated, except for the use of tributyl-(1-cyclobutylimidazol-4-yl)stannane (95 mg, 0.23 mmol) instead of tributyl-(3-methylpyrazin-2-yl)stannane in [Process-7] and the use of ethyl acetate:hexane=2:1 (v/v) instead of dichloromethane:methanol=20:1 (v/v) during purification, of Example 1 above, to obtain 5 mg (9% yield) of the title compound.

¹H-NMR (300 MHz, CDCl₃): δ 8.53 (d, J=1.2 Hz, 1H), 7.91 (s, 1H), 7.84 (d, J=8.4 Hz, 2H), 7.76-7.60 (m, 5H), 7.38 (d, J=1.1 Hz, 1H), 4.72-4.66 (m, 1H), 4.33-4.29 (m, 1H), 2.67 (d, J=5.4 Hz, 3H), 2.61-2.55 (m, 2H), 2.51-2.45 (m, 2H), 2.09-1.95 (m, 2H).

MS (ESI⁺, m/z): 475.2 [M+H]⁺

### Example 17: N-methyl-3-phenyl-1-(4-(trifluoromethyl)phenyl)-1H-indole-5-sulfonamide

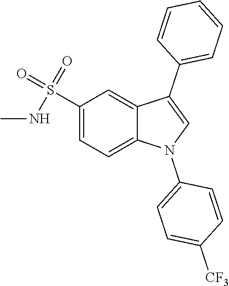 [SMILES: CNS(=O)(=O)c1ccc2c(c1)c(-c1ccccc1)cn2-c1ccc(C(F)(F)F)cc1]

The procedure of Example 3 was repeated except for the use of phenylboronic acid (20 mg, 0.16 mmol) instead of 1-methyl-3-(4,4,5,5-tetramethyl-1,3,2-dioxaborolan-2-yl) pyrazole in Example 3 above, to obtain 14 mg (28% yield) of the title compound.

¹H-NMR (300 MHz, CDCl₃): δ 8.51 (s, 1H), 7.86 (d, J=8.6 Hz, 2H), 7.99 (d, J=1.8 Hz, 1H), 7.77-7.61 (m, 5H), 7.54 (s, 1H), 7.51-7.49 (m, 2H), 7.42-7.39 (m, 1H), 4.28-4.23 (m, 1H), 2.66 (d, J=1.8 Hz, 3H).

MS (ESI⁺, m/z): 431.2 [M+H]⁺

### Example 18:1-(4-cyclohexylphenyl)-N-methyl-3-(1-methyl-1H-imidazol-4-yl)-1H-indole-5-sulfonamide

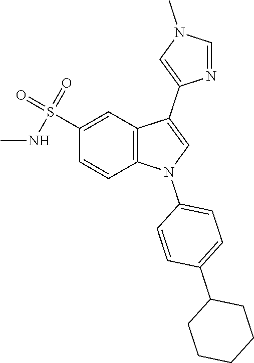 [SMILES: CNS(=O)(=O)c1ccc2c(c1)c(-c1cn(C)cn1)cn2-c1ccc(C2CCCCC2)cc1]

The procedure of Example 5 was repeated except for the use of 1-bromo-4-cyclohexyl-benzene (439 mg, 1.78 mmol) instead of 1-bromo-4-(trifluoromethoxy)benzene in Example 5 above, to obtain 18 mg (29% yield) of the title compound.

¹H-NMR (300 MHz, CDCl₃): δ 8.41 (s, 1H), 7.84-7.79 (m, 2H), 7.68-7.61 (m, 2H), 7.58 (s, 1H), 7.52-7.36 (m, 4H), 4.58-4.54 (m, 1H), 3.83 (s, 3H), 2.65-2.59 (m, 1H), 2.51 (s, 3H), 1.94-1.88 (m, 4H), 1.81-1.77 (m, 1H), 1.58-1.31 (m, 5H).

MS (ESI⁺, m/z): 449.2 [M+H]⁺

### Example 19: N,N-dimethyl-3-(1-methyl-1H-imidazol-4-yl)-1-(4-(trifluoromethyl)phenyl)-1H-indole-5-sulfonamide

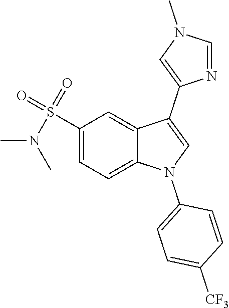 [SMILES: CN(C)S(=O)(=O)c1ccc2c(c1)c(-c1cn(C)cn1)cn2-c1ccc(C(F)(F)F)cc1]

The procedure of Example 1 was repeated, except for the use of 2 M dimethylamine tetrahydrofuran solution (17.3 mL, 34.7 mmol) instead of 2M methylamine tetrahydrofuran solution in [Process-2] and tributyl-(1-methylimidazol-4-yl)stannane (112 mg, 0.3 mmol) instead of tributyl-(3-methylpyrazin-2-yl)stannane, of Example 1 above, to obtain 19 mg (19% yield) of the title compound.

¹H-NMR (300 MHz, DMSO-d₆): δ 8.68 (s, 1H), 8.18 (s, 1H), 8.00-7.92 (m, 4H), 7.87-7.84 (m, 1H), 7.74 (s, 1H), 7.63-7.59 (m, 1H), 7.58 (s, 1H), 3.74 (s, 3H), 2.62 (s, 6H).

MS (ESI⁺, m/z): 449.2 [M+H]⁺

### Example 20:3-(3-fluoropyridin-2-yl)-N-methyl-1-(4-(trifluoromethyl)phenyl)-1H-indole-5-sulfonamide

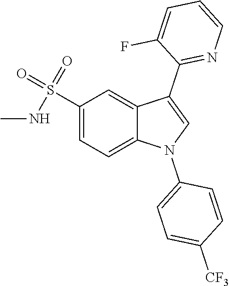 [SMILES: CNS(=O)(=O)c1ccc2c(c1)c(-c1ncccc1F)cn2-c1ccc(C(F)(F)F)cc1]

The procedure of Example 1 was repeated, except for the use of tributyl-(3-fluoro-2-pyridinyl)stannane (89 mg, 0.23 mmol) instead of tributyl-(3-methylpyrazin-2-yl)stannane in [Process-7] and the use of ethyl acetate:hexane=1:1 (v/v) instead of dichloromethane:methanol=20:1 (v/v) during purification, of Example 1 above, to obtain 19 mg (35% yield) of the title compound.

¹H-NMR (300 MHz, DMSO-d₆): δ 8.75 (s, 1H), 8.59 (d, J=4.8 Hz, 1H), 8.45 (s, 1H), 8.25 (s, 1H), 8.05-7.97 (m, 4H), 7.91-7.83 (m, 2H), 7.76-7.72 (m, 1H), 7.43-7.41 (m, 1H), 2.40 (d, J=4.8 Hz, 3H).

MS (ESI⁺, m/z): 450.3 [M+H]⁺

### Example 21: N-methyl-3-(1-methyl-1H-imidazol-4-yl)-1-(3-(trifluoromethyl)phenyl)-1H-indole-5-sulfonamide

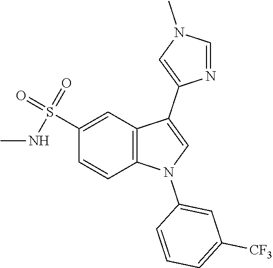 [SMILES: CNS(=O)(=O)c1ccc2c(c1)c(-c1cn(C)cn1)cn2-c1cccc(C(F)(F)F)c1]

The procedure of Example 5 was repeated except for the use of 3-bromobenzotrifluoride (490 mg, 2.14 mmol) instead of 1-bromo-4-(trifluoromethoxy)benzene in Example 5 above, to obtain 20 mg (22% yield) of the title compound.

¹H-NMR (300 MHz, DMSO-de): δ 8.73 (s, 1H), 8.16 (s, 1H), 8.04-8.02 (m, 2H), 7.88-7.86 (m, 2H), 7.66-7.65 (m, 2H), 7.64-7.63 (m, 1H), 7.55-7.54 (m, 1H), 7.35-7.34 (m, 1H), 3.74 (s, 3H), 2.40 (d, J=4.9 Hz, 3H).

MS (ESI⁺, m/z): 435.2 [M+H]⁺

### Example 22:1-(4-cyanophenyl)-N-methyl-3-(1-methyl-1H-imidazol-4-yl)-1H-indole-5-sulfonamide

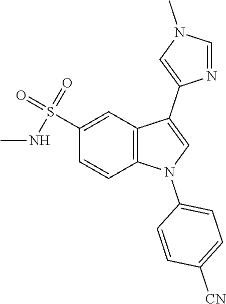 [SMILES: CNS(=O)(=O)c1ccc2c(c1)c(-c1cn(C)cn1)cn2-c1ccc(C#N)cc1]

The procedure of Example 5 was repeated except for the use of 4-iodobenzonitrile (500 mg, 2.14 mmol) instead of 1-bromo-4-(trifluoromethoxy)benzene in Example 5 above, to obtain 25 mg (50% yield) of the title compound.

¹H-NMR (300 MHz, DMSO-d₆): δ 8.73-8.72 (m, 1H), 8.16 (s, 1H), 8.10-8.07 (m, 2H), 7.93-7.83 (m, 3H), 7.74 (s, 1H), 7.68-7.64 (m, 1H), 7.56 (s, 1H), 7.39-7.34 (m, 1H), 3.75 (s, 3H), 2.40 (d, J=4.9 Hz, 3H).

MS (ESI⁺, m/z): 392.2 [M+H]⁺

### Example 23:3-(furan-3-yl)-N-methyl-1-(4-(trifluoromethyl)phenyl)-1H-indole-5-sulfonamide

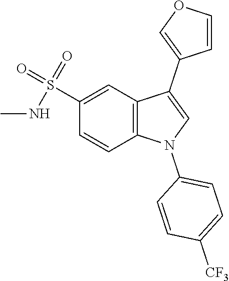 [SMILES: CNS(=O)(=O)c1ccc2c(c1)c(-c1ccoc1)cn2-c1ccc(C(F)(F)F)cc1]

The procedure of Example 3 was repeated except for the use of 3-furylboronic acid (21 mg, 0.18 mmol) instead of 1-methyl-3-(4,4,5,5-tetramethyl-1,3,2-dioxaboran-2-yl) pyrazole in Example 3 above, to obtain 13 mg (19% yield) of the title compound.

¹H-NMR (300 MHz, DMSO-d₆): δ 8.28-8.23 (m, 3H), 8.01-7.92 (m, 4H), 7.87-7.83 (m, 2H), 7.71-7.68 (m, 1H), 7.50-7.30 (m, 1H), 7.02 (s, 1H), 2.40 (d, J=4.8 Hz, 3H).

MS (ESI⁺, m/z): 421.2 [M+H]⁺

### Example 24: N-methyl-3-(5-methylfuran-2-yl)-1-phenyl-1H-indole-5-sulfonamide

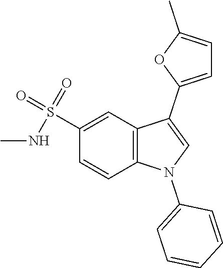 [SMILES: CNS(=O)(=O)c1ccc2c(c1)c(-c1ccc(C)o1)cn2-c1ccccc1]

The procedure of Example 10 was repeated except for the use of bromobenzene (339 mg, 2.14 mmol) instead of 4-bromobenzotrifluoride in Example 10 above, to obtain 5 mg (9% yield) of the title compound.

¹H-NMR (300 MHz, DMSO-d₆): δ 8.40 (s, 1H), 8.12 (s, 1H), 7.74-7.60 (m, 6H), 7.52-7.46 (m, 1H), 7.47-7.42 (m, 1H), 6.69-6.68 (m, 1H), 6.26-6.25 (m, 1H), 2.42 (s, 3H), 2.40 (d, J=4.8 Hz, 3H).

MS (ESI⁺, m/z): 367.2 [M+H]⁺

### Example 25:3-(2,3-difluorophenyl)-N-methyl-1-(4-(trifluoromethyl)phenyl)-1H-indole-5-sulfonamide

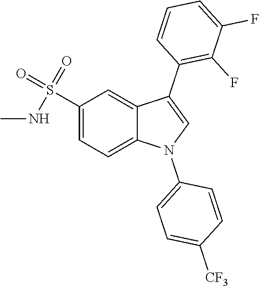 [SMILES: CNS(=O)(=O)c1ccc2c(c1)c(-c1cccc(F)c1F)cn2-c1ccc(C(F)(F)F)cc1]

The procedure of Example 3 was repeated except for the use of 2,3-difluorophenylboronic acid (32 mg, 0.21 mmol) instead of 1-methyl-3-(4,4,5,5-tetramethyl-1,3,2-dioxaborolan-2-yl) pyrazole in Example 3 above, to obtain 10 mg (14% yield) of the title compound.

¹H-NMR (300 MHz, CDCl₃): δ 8.39 (s, 1H), 7.88 (d, J=8.6 Hz, 2H), 7.81-7.67 (m, 5H), 7.49-7.44 (m, 1H), 7.24-7.18 (m, 2H), 4.29-4.25 (m, 1H), 2.68 (d, J=5.5 Hz, 3H).

MS (ESI⁺, m/z): 467.2 [M+H]⁺

### Example 26: N-methyl-3-(1-methyl-1H-imidazol-4-yl)-1-phenyl-1H-indole-5-sulfonamide

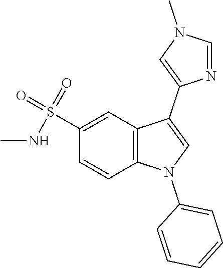 [SMILES: CNS(=O)(=O)c1ccc2c(c1)c(-c1cn(C)cn1)cn2-c1ccccc1]

The procedure of Example 5 was repeated except for the use of bromobenzene (339 mg, 2.14 mmol) instead of 1-bromo-4-(trifluoromethoxy)benzene in Example 5 above, to obtain 22 mg (35% yield) of the title compound.

¹H-NMR (300 MHz, DMSO-d₆): δ 8.69 (s, 1H), 8.03 (s, 1H), 7.71-7.60 (m, 7H), 7.53 (s, 1H), 7.53-7.52 (m, 1H), 7.49-7.46 (m, 1H), 3.74 (s, 3H), 2.40 (d, J=4.8 Hz, 3H).

MS (ESI⁺, m/z): 367.2 [M+H]⁺

### Example 27:1-(3-chloro-4-(trifluoromethyl)phenyl)-N-methyl-3-(1-methyl-1H-imidazol-4-yl)-1H-indole-5-sulfonamide

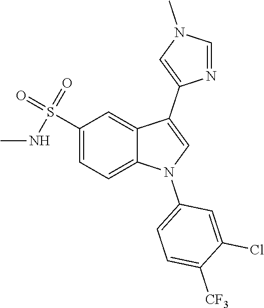 [SMILES: CNS(=O)(=O)c1ccc2c(c1)c(-c1cn(C)cn1)cn2-c1ccc(C(F)(F)F)c(Cl)c1]

The procedure of Example 5 was repeated except for the use of 4-bromo-2-chloro-1-(trifluoromethyl)benzene (566 mg, 2.14 mmol) instead of 1-bromo-4-(trifluoromethoxy)benzene in Example 5, to obtain 2.5 mg (3% yield) of the title compound.

¹H-NMR (300 MHz, CDCl₃): δ 8.51-8.50 (m, 1H), 7.91-7.87 (m, 2H), 7.80-7.66 (m, 3H), 7.58-7.56 (m, 2H), 7.33 (s, 1H), 4.63-4.61 (m, 1H), 3.80 (s, 3H), 2.66 (d, J=4.9 Hz, 3H).

MS (ESI⁺, m/z): 469.2 [M+H]⁺

### Example 28:3-(1-cyclopropyl-1H-imidazol-4-yl)-N-methyl-1-(4-(trifluoromethyl)phenyl)-1H-indole-5-sulfonamide

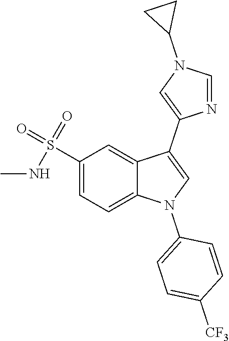 [SMILES: CNS(=O)(=O)c1ccc2c(c1)c(-c1cn(C3CC3)cn1)cn2-c1ccc(C(F)(F)F)cc1]

The procedure of Example 1 was repeated, except for the use of tributyl-(1-cyclopropylimidazol-4-yl)stannane (73 mg, 0.18 mmol) instead of tributyl-(3-methylpyrazin-2-yl)stannane in [Process-7] and the use of ethyl acetate:hexane=2:1 (v/v) instead of dichloromethane:methanol=20:1 (v/v) during purification, of Example 1 above, to obtain 3 mg (5% yield) of the title compound.

¹H-NMR (300 MHz, DMSO-d₆): δ 8.75 (d, J=1.6 Hz, 1H), 8.16 (s, 1H), 8.00-7.90 (m, 4H), 7.85-7.81 (m, 2H), 7.67-7.62 (m, 2H), 7.37-7.35 (m, 1H), 3.62-3.55 (m, 1H), 2.41 (d, J=4.8 Hz, 3H), 1.03-0.99 (m, 4H).

MS (ESI⁺, m/z): 461.1 [M+H]⁺

### Example 29:3-(1H-imidazol-4-yl)-N-methyl-1-(4-(trifluoromethyl)phenyl)-1H-indole-5-sulfonamide

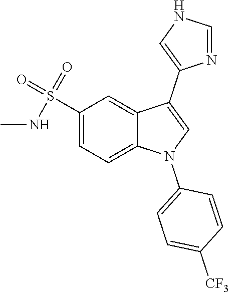 [SMILES: CNS(=O)(=O)c1ccc2c(c1)c(-c1c[nH]cn1)cn2-c1ccc(C(F)(F)F)cc1]

The procedure of Example 1 was repeated, except for the use of t-butyl 4-(tributylstannyl)-1H-imidazole-1-carboxylate (150 mg, 0.33 mmol) instead of tributyl-(3-methylpyrazin-2-yl)stannane in [Process-7] and the use of ethyl acetate:hexane=4:1 (v/v) instead of dichloromethane:methanol=20:1 (v/v) during purification, of Example 1 above, to obtain 14 mg (20% yield) of the title compound.

¹H-NMR (300 MHz, DMSO-d₆): δ 12.2 (brs, 1H), 8.73 (s, 1H), 8.16 (s, 1H), 7.99-7.90 (m, 4H), 7.84-7.82 (m, 2H), 7.67-7.65 (m, 1H), 7.57 (s, 1H), 7.36-7.34 (m, 1H), 2.41 (d, J=4.8 Hz, 3H).

MS (ESI⁺, m/z): 421.2 [M+H]⁺

### Example 30:3-(1-(2-fluorobenzyl)-1H-imidazol-4-yl)-N-methyl-1-(4-(trifluoromethyl)phenyl)-1H-indole-5-sulfonamide

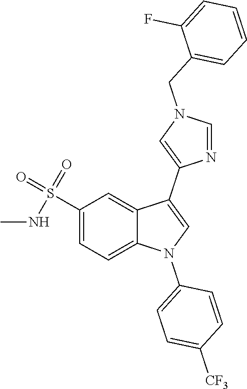 [SMILES: CNS(=O)(=O)c1ccc2c(c1)c(-c1cn(Cc3ccccc3F)cn1)cn2-c1ccc(C(F)(F)F)cc1]

The procedure of Example 1 was repeated, except for the use of tributyl-[1-[(2-fluorophenyl)methyl]imidazol-4-yl]stannane (86 mg, 0.18 mmol) instead of tributyl-(3-methylpyrazin-2-yl)stannane in [Process-7] and the use of ethyl acetate:hexane=1:1 (v/v) instead of dichloromethane:methanol=20:1 (v/v) during purification, of Example 1 above, to obtain 9 mg (13% yield) of the title compound.

¹H-NMR (300 MHz, CDCl₃): δ 8.53 (d, J=1.4 Hz, 1H), 7.89 (s, 1H), 7.83 (d, J=8.4 Hz, 2H), 7.76-7.72 (m, 1H), 7.67-7.64 (m, 4H), 7.36-7.35 (m, 2H), 7.20-7.11 (m, 3H), 5.27 (s, 2H), 4.32-4.30 (m, 1H), 2.66 (d, J=5.5 Hz, 3H).

MS (ESI⁺, m/z): 529.3 [M+H]⁺

### Example 31:3-(furan-2-yl)-N-methyl-1-(4-(trifluoromethyl)phenyl)-1H-indole-5-sulfonamide

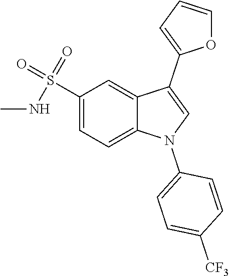 [SMILES: CNS(=O)(=O)c1ccc2c(c1)c(-c1ccco1)cn2-c1ccc(C(F)(F)F)cc1]

The procedure of Example 3 was repeated except for the use of 2-(2-furyl)-4,4,5,5-tetramethyl-1,3,2-dioxaborolan-2-yl) pyrazole (31 mg, 0.16 mmol) instead of 1-methyl-3-(4,4,5,5-tetramethyl-1,3,2-dioxaborolane in Example 3 above, to obtain 16 mg (32% yield) of the title compound.

¹H-NMR (300 MHz, DMSO-da): δ 8.47 (d, J=1.4 Hz, 1H), 8.35 (s, 1H), 8.02-7.94 (m, 4H), 7.87-7.83 (m, 2H), 7.71 (dd, J=8.8, 1.7 Hz, 1H), 7.50-7.41 (m, 1H), 6.86 (d, J=3.9 Hz, 1H), 6.67 (d, J=1.9 Hz, 1H), 2.41 (s, 3H).

MS (ESI⁺, m/z): 421.2 [M+H]⁺

### Example 32: N-methyl-3-(2-methyloxazol-5-yl)-1-(4-(trifluoromethyl)phenyl)-1H-indole-5-sulfonamide

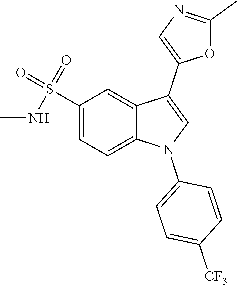

The procedure of Example 3 was repeated except for the use of 2-methyl-5-(4,4,5,5-tetramethyl-1,3,2-dioxaborolan-2-yl) oxazole (27 mg, 0.13 mmol) instead of 1-methyl-3-(4,4,5,5-tetramethyl-1,3,2-dioxaborolan-2-yl) pyrazole in Example 3 above, to obtain 4 mg (8% yield) of the title compound.

¹H-NMR (300 MHz, DMSO-d₆): δ 8.37-8.36 (m, 2H), 8.02-7.95 (m, 4H), 7.89-7.86 (m, 1H), 7.75-7.72 (m, 1H), 7.46 (s, 2H), 2.53 (s, 3H), 2.42 (d, J=3.1 Hz, 3H).

MS (ESI⁺, m/z): 436.3 [M+H]⁺

### Example 33: N-methyl-3-(1-methyl-1H-imidazol-4-yl)-1-(4-(trifluoromethyl)phenyl)-1H-indole-6-sulfonamide

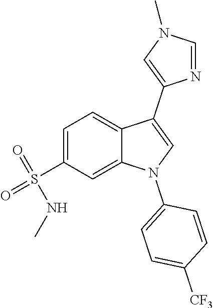 [SMILES: CNS(=O)(=O)c1ccc2c(-c3cn(C)cn3)cn(-c3ccc(C(F)(F)F)cc3)c2c1]

### [Process-1] Preparation of indoline-6-sulfonic acid

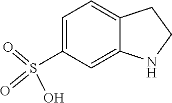 [SMILES: O=S(=O)(O)c1ccc2c(c1)NCC2]

Indoline (10 g, 83.9 mmol) was added dropwise to 46 mL of sulfuric acid at 0° C. to 5° C. and stirred at 135° C. for one hour. After confirming the completion of the reaction, the mixture was cooled to room temperature, and the formed solid was filtered to synthesize 6.7 g (40% yield) of indoline-6-sulfonic acid.

¹H-NMR (300 MHz, DMSO-d₆): δ 7.63-7.61 (m, 2H), 7.43-7.41 (m, 1H), 3.77-3.72 (m, 2H), 3.22-3.17 (m, 2H).

### [Process-2] Preparation of 1-acetylindoline-6-sulfonic acid

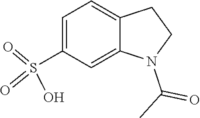 [SMILES: CC(=O)N1CCc2ccc(S(=O)(=O)O)cc21]

Indoline-6-sulfonic acid (4.6 g, 23.1 mmol) was added to acetic anhydride (3.3 mL, 34.6 mmol) and pyridine (11.2 mL, 138.5 mmol) and stirred at 100° C. overnight. After completion of the reaction, the mixture was cooled to room temperature, ethyl acetate was added, and the mixture was washed with water. The organic layer was dried over anhydrous sodium sulfate, filtered under reduced pressure, and the filtered organic layer was concentrated under reduced pressure to synthesize 4.1 g (74% yield) of 1-acetylindoline-6-sulfonic acid.

¹H-NMR (300 MHz, DMSO-d₆): δ 8.33 (s, 1H), 7.28-7.25 (m, 1H), 7.16-7.14 (m, 1H), 4.12-4.08 (m, 2H), 3.13-3.08 (m, 2H), 2.16 (s, 3H).

### [Process-3] Preparation of N-methylindoline-6-sulfonamide

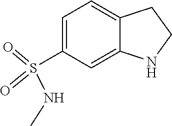

1-acetylindoline-6-sulfonic acid (4 g, 16.6 mmol) was dissolved in 50 ml of acetonitrile, dimethylformamide catalytic amount and POCl₃ (8 mL, 85 mmol) were added thereto. The mixture was refluxed and heated for 1 hour, concentrated, and then placed on ice, and the formed solid was filtered to obtain 1-acetylindoline-6-sulfonyl chloride as a brown solid, which was used in the next process without further purification. 1-acetylindoline-6-sulfonyl chloride was dissolved in dichloromethane, 2M methylamine (23 mL, 69.3 mmol) was added thereto, and the mixture was stirred at room temperature for 3 hours. After confirming the completion of the reaction, the solvent was removed by reducing the pressure, and then 40 mL of methanol and concentrated HCl (3 mL, 92.4 mmol) were added thereto and stirred at 80° C. After confirming the completion of the reaction, ethyl acetate was added thereto and washed with water. The organic layer was dried over anhydrous sodium sulfate and filtered under reduced pressure, and the filtered organic layer was concentrated under reduced pressure and purified with dichloromethane:methanol=10:1 (v/v) to obtain 2.6 g (53% yield) of the title compound.

¹H-NMR (300 MHz, CDCl₃): δ 7.19-7.18 (m, 2H), 7.04 (s, 1H), 3.66 (t, J=8.5 Hz, 2H), 3.01 (t, J=8.5 Hz, 2H), 2.66 (d, J=3.3 Hz, 3H).

### [Process-4] Preparation of N-methyl-1H-indole-6-sulfonamide

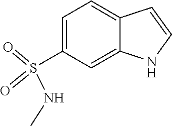

N-methylindoline-6-sulfonamide (1.8 g, 8.5 mmol) prepared in [Process-3] above was dissolved in 20 mL of dichloromethane, 2,3-dichloro-5,6-dicyano-1,4-benzoquinone (DDQ, 1.9 g, 8.5 mmol) was added thereto, and the reaction solution was stirred at room temperature overnight. After completion of the reaction, the reaction solution was filtered through CELITE and washed with dichloromethane. After concentration under reduced pressure, the obtained residue was purified by MPLC (ethyl acetate:hexane=1:10 (v/v)) to obtain 1.9 g (58% yield) of the title compound.

¹H-NMR (300 MHz, CDCl₃): δ 8.10 (s, 1H), 7.70-7.56 (m, 1H), 7.55-7.52 (m, 1H), 7.40-7.38 (m, 1H), 6.57-6.56 (m, 1H), 4.99-4.97 (m, 1H), 2.55 (d, J=5.3 Hz, 3H).

### [Process-5] Preparation of N-methyl-3-(1-methyl-1H-imidazol-4-yl)-1-(4-(trifluoromethyl)phenyl)-1H-indole-6-sulfonamide

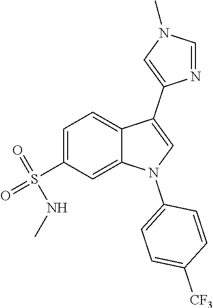 [SMILES: CNS(=O)(=O)c1ccc2c(-c3cn(C)cn3)cn(-c3ccc(C(F)(F)F)cc3)c2c1]

The procedure of Example 2 was repeated except for the use of N-methyl-1H-indole-6-sulfonamide (1 g, 4.75 mmol) instead of N-methyl-1H-indole-5-sulfonamide in Example 2 above, to obtain 18 mg (16% yield) of the title compound.

¹H-NMR (300 MHz, DMSO-d₆): δ 8.39-8.28 (m, 1H), 8.37 (s, 1H), 8.04-8.02 (m, 3H), 7.93-7.90 (m, 2H), 7.72 (s, 1H), 7.65-7.62 (m, 2H), 7.38-7.36 (m, 1H), 3.73 (s, 3H), 2.40 (d, J=6.0 Hz, 3H).

MS (ESI⁺, m/z): 435.1 [M+H]⁺

### Example 34: N-methyl-3-(5-(trifluoromethyl) furan-2-yl)-1-(4-(trifluoromethyl)phenyl)-1H-indole-5-sulfonamide

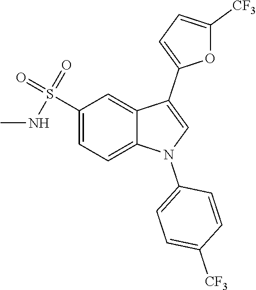 [SMILES: CNS(=O)(=O)c1ccc2c(c1)c(-c1ccc(C(F)(F)F)o1)cn2-c1ccc(C(F)(F)F)cc1]

The procedure of Example 3 was repeated except for the use of 4,4,5,5-tetramethyl-2-(5-(trifluoromethyl) furan-2-yl)-1,3,2-dioxaborolane (68 mg, 0.25 mmol) instead of 1-methyl-3-(4,4,5,5-tetramethyl-1,3,2-dioxaborolan-2-yl) pyrazole in Example 3 above, to obtain 30 mg (25% yield) of the title compound.

¹H-NMR (300 MHz, DMSO-de): δ 8.55 (s, 1H), 8.45 (d, J=1.3 Hz, 1H), 8.03-7.97 (m, 4H), 7.90-7.87 (m, 1H), 7.77-7.73 (m, 1H), 7.48-7.42 (m, 2H), 7.07 (d, J=3.6 Hz, 1H), 2.43 (d, J=2.9 Hz, 3H).

MS (ESI⁺, m/z): 489.4 [M+H]⁺

### Example 35: N-methyl-3-(1-(oxetan-3-yl)-1H-imidazol-4-yl)-1-(4-(trifluoromethyl)phenyl)-1H-indole-5-sulfonamide

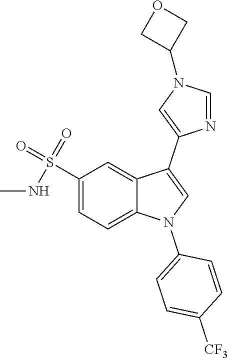 [SMILES: CNS(=O)(=O)c1ccc2c(c1)c(-c1cn(C3COC3)cn1)cn2-c1ccc(C(F)(F)F)cc1]

The procedure of Example 1 was repeated except for the use of 1-(oxetan-3-yl)-4-(tributylstannyl)-1H-imidazole (209 mg, 0.51 mmol) instead of tributyl-(3-methylpyrazin-2-yl)stannane in [Process-7] of Example 1 above, to obtain 5 mg (4% yield) of the title compound.

¹H-NMR (300 MHz, CDCl₃): δ 8.56 (s, 1H), 7.92 (s, 1H), 7.86-7.84 (m, 2H), 7.78-7.75 (m, 2H), 7.70-7.69 (m, 2H), 7.67-7.66 (m, 1H), 7.61-7.60 (m, 1H), 5.40-5.39 (m, 1H), 5.19 (t, J=7.1 Hz, 2H), 5.00-4.96 (m, 2H), 4.35-4.33 (m, 1H), 2.69 (d, J=5.5 Hz, 3H).

MS (ESI⁺, m/z): 477.2 [M+H]⁺

### Example 36:3-(5-chlorofuran-2-yl)-N-methyl-1-(4-(trifluoromethyl)phenyl)-1H-indole-5-sulfonamide

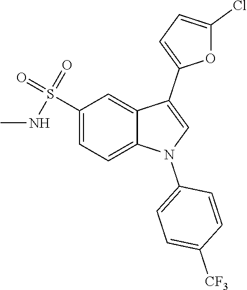 [SMILES: CNS(=O)(=O)c1ccc2c(c1)c(-c1ccc(Cl)o1)cn2-c1ccc(C(F)(F)F)cc1]

The procedure of Example 3 was repeated except for the use of 2-(5-chloro-2-furyl)-4,4,5,5-tetramethyl-1,3,2-dioxaborolane (74 mg, 0.32 mmol) instead of 1-methyl-3-(4,4,5,5-tetramethyl-1,3,2-dioxaborolan-2-yl) pyrazole in Example 3 above, to obtain 4 mg (3% yield) of the title compound.

¹H-NMR (300 MHz, DMSO-d₆): δ 8.39 (s, 1H), 8.02-7.84 (m, 6H), 7.74 (d, J=1.6 Hz, 1H), 7.47-7.45 (m, 1H), 6.94 (d, J=3.4 Hz, 1H), 6.69 (d, J=3.4 Hz, 1H), 2.42 (d, J=4.4 Hz, 3H).

MS (ESI⁺, m/z): 455.0 [M+H]⁺

### Example 37: N-methyl-3-(1-(2,2,2-trifluoroethyl)-1H-imidazol-4-yl)-1-(4-(trifluoromethyl)phenyl)-1H-indole-5-sulfonamide

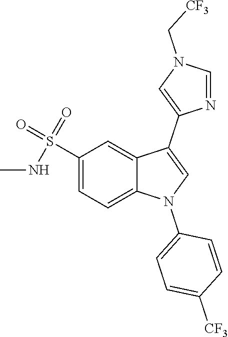 [SMILES: CNS(=O)(=O)c1ccc2c(c1)c(-c1cn(CC(F)(F)F)cn1)cn2-c1ccc(C(F)(F)F)cc1]

The procedure of Example 1 was repeated, except for the use of tributyl-[1-[(2-fluoroethyl) imidazol-4-yl]stannane (158 mg, 0.36 mmol) instead of tributyl-(3-methylpyrazin-2-yl)stannane in [Process-7] and the use of ethyl acetate:hexane=2:1 (v/v) instead of dichloromethane:methanol=20:1 (v/v) during purification, of Example 1 above, to obtain 30 mg (26% yield) of the title compound.

¹H-NMR (300 MHz, DMSO-de): δ 8.73 (d, J=1.4 Hz, 1H), 8.26 (s, 1H), 8.00-7.92 (m, 5H), 7.86 (d, J=8.8 Hz, 1H), 7.69-7.65 (m, 2H), 7.36-7.35 (m, 1H), 5.18-5.11 (m, 2H), 2.41 (d, J=5.1 Hz, 3H).

MS (ESI⁺, m/z): 503.4 [M+H]⁺

### Example 38:3-(1-(2-methoxyethyl)-1H-imidazol-4-yl)-N-methyl-1-(4-(trifluoromethyl)phenyl)-1H-indole-5-sulfonamide

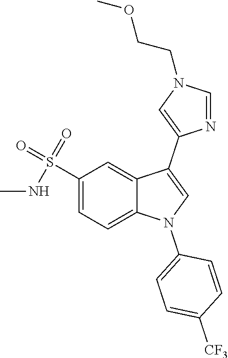 [SMILES: CNS(=O)(=O)c1ccc2c(c1)c(-c1cn(CCOC)cn1)cn2-c1ccc(C(F)(F)F)cc1]

The procedure of Example 1 was repeated except for the use of tributyl-[1-(2-methoxyethyl)imidazol-4-yl)stannane (316 mg, 0.76 mmol) instead of tributyl-(3-methylpyrazin-2-yl)stannane in [Process-7] of Example 1 above, to obtain 30 mg (24% yield) of the title compound.

¹H-NMR (300 MHz, DMSO-d₆): δ 8.74 (s, 1H), 8.16 (s, 1H), 7.99-7.91 (m, 4H), 7.85-7.82 (m, 1H), 7.77 (s, 1H), 7.68-7.64 (m, 1H), 7.60 (s, 1H), 7.36-7.34 (m, 1H), 4.22 (t, J=5.0 Hz, 2H), 3.67 (t, J=5.2 Hz, 2H), 3.29 (s, 3H), 2.41 (d, J=5.0 Hz, 3H).

MS (ESI⁺, m/z): 479.2 [M+H]⁺

### Example 39: N-methyl-3-(1-methyl-1H-imidazol-4-yl)-1-(p-tolyl)-1H-indole-5-sulfonamide

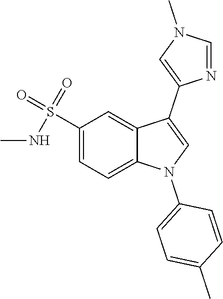 [SMILES: CNS(=O)(=O)c1ccc2c(c1)c(-c1cn(C)cn1)cn2-c1ccc(C)cc1]

The procedure of Example 1 was repeated, except for the use of 1-bromo-4-methyl-benzene (747 mg, 4.28 mmol) instead of 4-bromobenzotrifluoride in [Process-5], the use of tributyl-(1-methylimidazol-4-yl)stannane (132 mg, 0.35 mmol) instead of tributyl-(3-methylpyrazin-2-yl)stannane in [Process-7], and the use of ethyl acetate:hexane=2:1 (v/v) instead of dichloromethane:methanol=20:1 (v/v) during purification, of Example 1 above, to obtain 16 mg (23% yield) of the title compound.

¹H-NMR (300 MHz, DMSO-di): δ 8.69 (d, J=1.2 Hz, 1H), 7.98 (s, 1H), 7.71-7.70 (m, 1H), 7.66-7.52 (m, 5H), 7.43-7.40 (m, 2H), 7.30-7.28 (m, 1H), 3.74 (s, 3H), 2.41-2.39 (m, 6H).

MS (ESI⁺, m/z): 381.2 [M+H]⁺

### Example 40:1-(4-(t-butyl)phenyl)-N-methyl-3-(1-methyl-1H-imidazol-4-yl)-1H-indole-5-sulfonamide

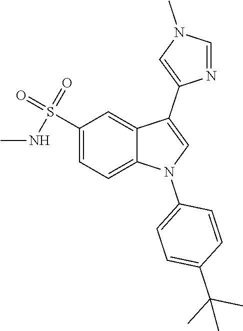 [SMILES: CNS(=O)(=O)c1ccc2c(c1)c(-c1cn(C)cn1)cn2-c1ccc(C(C)(C)C)cc1]

The procedure of Example 1 was repeated, except for the use of 1-bromo-4-t-butyl-benzene (979 mg, 4.41 mmol) instead of 4-bromobenzotrifluoride in [Process-5], the use of tributyl-(1-methylimidazol-4-yl)stannane (176 mg, 0.47 mmol) instead of tributyl-(3-methylpyrazin-2-yl)stannane in [Process-7], and the use of ethyl acetate:hexane=2:1 (v/v) instead of dichloromethane:methanol=20:1 (v/v) during purification, of Example 1 above, to obtain 50 mg (48% yield) of the title compound.

¹H-NMR (300 MHz, DMSO-d₆): δ 8.70 (d, J=1.4 Hz, 1H), 8.01 (s, 1H), 7.73-7.57 (m, 7H), 7.52 (d, J=1.2 Hz, 1H), 7.31 (s, 1H), 3.75 (s, 3H), 2.41 (d, J=4.6 Hz, 3H), 1.38 (s, 9H).

MS (ESI⁺, m/z): 423.2 [M+H]⁺

### Example 41: N-methyl-3-(oxazol-5-yl)-1-(4-(trifluoromethyl)phenyl)-1H-indole-5-sulfonamide

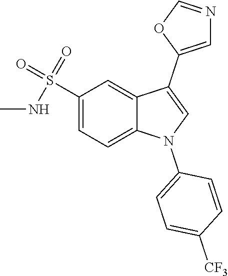 [SMILES: CNS(=O)(=O)c1ccc2c(c1)c(-c1cnco1)cn2-c1ccc(C(F)(F)F)cc1]

The procedure of Example 3 was repeated, except for the use of 5-(4,4,5,5-tetramethyl-1,3,2-dioxaboran-2-yl) oxazole (189 mg, 0.97 mmol) instead of 1-methyl-3-(4,4,5,5-tetramethyl-1,3,2-dioxaborolan-2-yl) pyrazole and the use of ethyl acetate:hexane=2:1 (v/v) instead of ethyl acetate:hexane=1:1 (v/v) during purification, in Example 3 above, to obtain 16 mg (5% yield) of the title compound.

¹H-NMR (300 MHz, CDCl₃): δ 8.52 (s, 1H), 7.99 (s, 1H), 7.90-7.80 (m, 4H), 7.69-7.65 (m, 3H), 7.42 (s, 1H), 4.34 (brs, 1H), 2.71 (d, J=5.5 Hz, 3H).

MS (ESI⁺, m/z): 422.1 [M+H]⁺

### Example 42:3-(1-cyclopropyl-1H-imidazol-4-yl)-1-(2-fluoro-4-(trifluoromethyl)phenyl)-N-methyl-1H-indole-5-sulfonamide

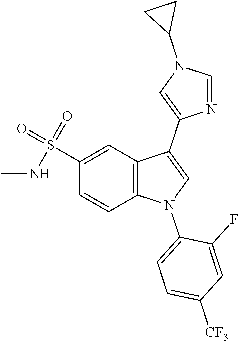 [SMILES: CNS(=O)(=O)c1ccc2c(c1)c(-c1cn(C3CC3)cn1)cn2-c1ccc(C(F)(F)F)cc1F]

The procedure of Example 28 was repeated except for the use of 1-bromo-2-fluoro-4-(trifluoromethyl)benzene (367 mg, 1.42 mmol) instead of 4-bromobenzotrifluoride in Example 28 above, to obtain 11 mg (6% yield) of the title compound.

¹H-NMR (300 MHz, CDCl₃): δ 8.55 (s, 1H), 7.84 (s, 1H), 7.83-7.77 (m, 4H), 7.63-7.40 (m, 2H), 4.46-4.44 (m, 1H), 3.49-3.44 (m, 1H), 2.71 (t, J=5.4 Hz, 1H), 2.70 (d, J=5.4 Hz, 3H), 0.87-0.85 (m, 4H).

MS (ESI⁺, m/z): 479.1 [M+H]⁺

### Example 43:3-(1-cyclopropyl-1H-imidazol-4-yl)-N-methyl-1-(4-(trifluoromethyl)phenyl)-1H-pyrrolo[2,3-b]pyridine-5-sulfonamide

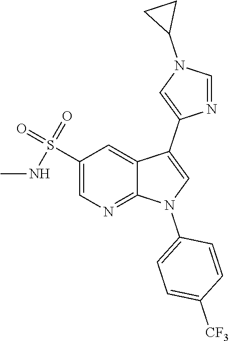 [SMILES: CNS(=O)(=O)c1cnc2c(c1)c(-c1cn(C3CC3)cn1)cn2-c1ccc(C(F)(F)F)cc1]

### [Process-1] Preparation of 5-bromo-1-(4-(trifluoromethyl)phenyl)-1H-pyrrolo[2,3-b]pyridine

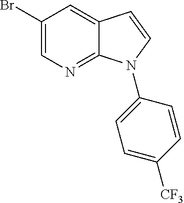 [SMILES: FC(F)(F)c1ccc(-n2ccc3cc(Br)cnc32)cc1]

5-bromo-1H-pyrrolo[2,3-b]pyridine (1 g, 5.07 mmol) was dissolved in 5 mL of 1,4-dioxane, followed by addition of racemic trans-1,2-diaminocyclohexane (0.013 mL, 0.10 mmol), copper iodide (1.9 mg, 0.01 mmol), potassium triphosphate (538 mg, 2.54 mmol) and 1-iodo-4-(trifluoromethyl)benzene (1.42 g, 5.07 mmol) sequentially, and the reaction solution was refluxed and stirred under nitrogen overnight. After completion of the reaction, the mixture was cooled to room temperature, filtered through CELITE, and washed with ethyl acetate. The residue obtained after concentration under reduced pressure was subjected to extraction with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:9 (v/v) to obtain 800 mg (46% yield) of the title compound.

¹H-NMR (300 MHz, CDCl₃): δ 8.39 (d, J=2.2 Hz, 1H), 8.09 (d, J=2.2 Hz, 1H), 7.93-7.90 (m, 2H), 7.78-7.75 (m, 2H), 7.55 (d, J=3.7 Hz, 1H), 6.62 (d, J=3.7 Hz, 1H).

### [Process-2] Preparation of 5-(benzylthio)-1-(4-(trifluoromethyl)phenyl)-1H-pyrrolo[2,3-b]pyridine

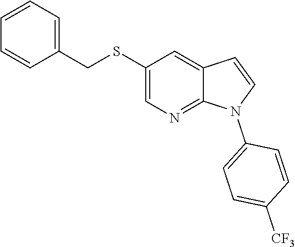 [SMILES: FC(F)(F)c1ccc(-n2ccc3cc(SCc4ccccc4)cnc32)cc1]

The 5-bromo-1-(4-(trifluoromethyl)phenyl)-1H-pyrrolo[2,3-b]pyridine (800 mg, 2.34 mmol) obtained in [Process-1] above was dissolved in 10 mL of toluene, followed by addition of benzyl mercaptan (0.27 mL, 2.34 mmol), [tris(dibenzylideneacetone)dipalladium(0)] (214 mg, 0.23 mmol), xantphos (271 mg, 0.47 mmol) and N,N-diisopropylethylamine (1.22 mL, 7.03 mmol), and the reaction solution was refluxed and stirred overnight. After confirming the completion of the reaction, the mixture was cooled to room temperature and subjected to extraction with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:9 (v/v)) to obtain 900 mg (98% yield) of the title compound.

¹H-NMR (300 MHz, CDCl₃): δ 8.36 (s, 1H), 7.98-7.92 (m, 3H), 7.81-7.78 (m, 2H), 7.56 (d, J=3.7 Hz, 1H), 7.29-7.19 (m, 5H), 6.62 (d, J=3.7 Hz, 1H), 4.07 (s, 2H).

### [Process-3] Preparation of 1-(4-(trifluoromethyl)phenyl)-1H-pyrrolo[2,3-b]pyridine-5-sulfonyl chloride

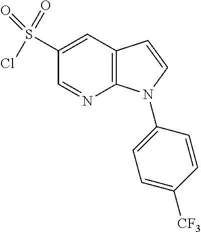 [SMILES: O=S(=O)(Cl)c1cnc2c(ccn2-c2ccc(C(F)(F)F)cc2)c1]

5-(benzylthio)-1-(4-(trifluoromethyl)phenyl)-1H-pyrrolo[2,3-b]pyridine (900 mg, 2.34 mmol) obtained in [Process-2] above was dissolved in 19.8 mL of acetic acid:water=10:1 (v/v), and N-chlorosuccinimide (937 mg, 7.02 mmol) was slowly added thereto under cooling to 0° C. to 5° C. The reaction mixture was stirred at 0° C. to 5° C. for 15 minutes. The temperature was raised to room temperature, and then mixture was stirred overnight. After confirming the completion of the reaction, the mixture was subjected to extraction with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:9 (v/v)) to obtain 380 mg (45% yield) of the title compound.

¹H-NMR (300 MHz, CDCl₃): δ 9.01 (d, J=2.3 Hz, 1H), 8.66 (d, J=2.3 Hz, 1H), 7.95-7.92 (m, 2H), 7.86-7.83 (m, 2H), 7.77 (d, J=3.7 Hz, 1H), 6.92 (d, J=3.7 Hz, 1H).

### [Process-4] Preparation of N-methyl-1-(4-(trifluoromethyl)phenyl)-1H-pyrrolo[2,3-b]pyridine-5-sulfonamide

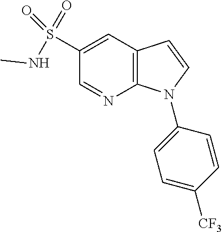 [SMILES: CNS(=O)(=O)c1cnc2c(ccn2-c2ccc(C(F)(F)F)cc2)c1]

1-(4-(trifluoromethyl)phenyl)-1H-pyrrolo[2,3-b]pyridine-5-sulfonyl chloride (380 mg, 1.05 mmol) obtained in [Process-3] above was dissolved in 7.6 mL of dichloromethane, and 2M methylamine tetrahydrofuran solution (4.2 mL, 2.10 mmol) was added dropwise thereto and stirred at room temperature overnight. After completion of the reaction, the mixture was subjected to extraction with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:2 (v/v)) to obtain 370 mg (44% yield) of the title compound.

¹H-NMR (300 MHz, CDCl₃): δ 8.85 (d, J=2.1 Hz, 1H), 8.50 (d, J=2.1 Hz, 1H), 7.96-7.93 (m, 2H), 7.84-7.81 (m, 2H), 7.70 (d, J=3.7 Hz, 1H), 6.83 (d, J=3.7 Hz, 1H), 4.41-4.35 (m, 1H), 2.73 (d, J=5.4 Hz, 3H).

### [Process-5] Preparation of 3-bromo-N-methyl-1-(4-(trifluoromethyl)phenyl)-1H-pyrrolo[2,3-b]pyridine-5-sulfonamide

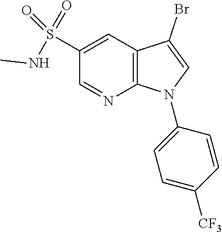 [SMILES: CNS(=O)(=O)c1cnc2c(c1)c(Br)cn2-c1ccc(C(F)(F)F)cc1]

N-methyl-1-(4-(trifluoromethyl)phenyl)-1H-pyrrolo[2,3-b]pyridine-5-sulfonamide (370 mg, 1.04 mmol) obtained in [Process-4] above was dissolved in 7.4 mL of dimethylformamide, and N-bromosuccinimide (198 mg, 1.09 mmol) was slowly added dropwise thereto. The reaction solution was stirred at room temperature for 1.5 hours, to complete the reaction, and 100 ml of water was added thereto, followed by extraction three times with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:3 (v/v)) to obtain 190 mg (42% yield) of the title compound.

¹H-NMR (300 MHz, CDCl₃): δ 8.87 (s, 1H), 8.46 (d, J=2.1 Hz, 1H), 7.92-7.89 (m, 2H), 7.84-7.81 (m, 2H), 7.74 (s, 1H), 4.50-4.48 (m, 1H), 2.74 (d, J=5.4 Hz, 3H).

### [Process-6] 3-(1-cyclo-1H-imidazol-4-yl)-N-methyl-1-(4-(trifluoromethyl)phenyl)-1H-pyrrolo[2,3-b]pyridine-5-sulfonamide

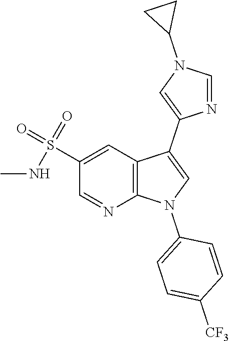 [SMILES: CNS(=O)(=O)c1cnc2c(c1)c(-c1cn(C3CC3)cn1)cn2-c1ccc(C(F)(F)F)cc1]

3-bromo-N-methyl-1-(4-(trifluoromethyl)phenyl)-1H-pyrrolo[2,3-b]pyridin-5-sulfonamide (70 mg, 0.16 mmol) obtained in [Process-5] above was dissolved in 1.4 mL of dimethylacetamide, followed by addition of copper chloride (3.2 mg, 0.03 mmol), cesium fluoride (74 mg, 0.48 mmol), [1,1′-bis(diphenylphosphino)bisferrocenyl]palladium dichloride (23 mg, 0.03 mmol), and tributyl-(1-cyclopropylimidazol-4-yl)stannane (103 mg, 0.25 mmol). Then, the reaction solution was stirred at 100° C. overnight. After completion of the reaction, water was added and extraction was performed thereon three times with ethyl acetate. The organic layer thus was dried over anhydrous sodium sulfate, filtered under reduced pressure, and the filtered organic layer was concentrated under reduced pressure, and the obtained residue was purified by MPLC (dichloromethane:methanol=20:1 (v/v)) to obtain 4 mg (5% yield) of the title compound.

¹H-NMR (300 MHz, DMSO-d₆): δ 9.01 (d, J=2.2 Hz, 1H), 8.74 (d, J=2.2 Hz, 1H), 8.54 (s, 1H), 8.26-8.25 (m, 2H), 8.12 (s, 1H), 8.00-7.97 (m, 2H), 7.90-7.83 (m, 1H), 7.63-7.61 (m, 1H), 3.69-3.61 (m, 1H), 2.45 (s, 3H), 1.08-1.02 (m, 4H).

MS (ESI⁺, m/z): 462.2 [M+H]⁺

### Example 44:3-(1-cyclopropyl-1H-imidazol-4-yl)-N-methyl-1-(4-(trifluoromethyl)phenyl)-1H-indazol-5-sulfonamide

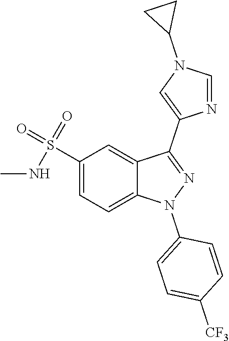 [SMILES: CNS(=O)(=O)c1ccc2c(c1)c(-c1cn(C3CC3)cn1)nn2-c1ccc(C(F)(F)F)cc1]

The procedure of Example 43 was repeated except for the use of 5-bromo-1H-indazole (5 g, 25.4 mmol) instead of 5-bromo-1H-pyrrolo[2,3-b]pyridine in [Process-1] of Example 43 above, to obtain 11 mg (9% yield) of the title compound.

¹H-NMR (300 MHz, DMSO-d₆): δ 9.01 (s, 1H), 8.34-8.21 (m, 3H), 8.12-8.01 (m, 3H), 7.91-7.64 (m, 2H), 7.52 (s, 1H), 3.69-3.61 (m, 1H), 2.28-2.27 (m, 3H), 1.10-0.98 (m, 4H).

MS (ESI⁺, m/z): 462.1 [M+H]⁺

### Example 45:3-(6,7-dihydro-5H-pyrrolo[1,2-a]imidazol-2-yl)-N-methyl-1-(4-(trifluoromethyl)phenyl)-1H-indole-5-sulfonamide

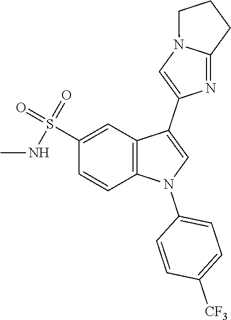 [SMILES: CNS(=O)(=O)c1ccc2c(c1)c(-c1cn3c(n1)CCC3)cn2-c1ccc(C(F)(F)F)cc1]

The procedure of Example 3 was repeated except for the use of 2-(4,4,5,5-tetramethyl-1,3,2-dioxaboran-2-yl)-6,7-dihydro-5H-pyrrolo[1,2-a]imidazole (43 mg, 0.17 mmol) instead of 1-methyl-3-(4,4,5,5-tetramethyl-1,3,2-dioxaborolan-2-yl) pyrazole in Example 3 above, to obtain 26 mg (34% yield) of the title compound.

¹H-NMR (300 MHz, DMSO-d₆): δ 8.69 (d, J=1.4 Hz, 1H), 8.13 (s, 1H), 7.99-7.91 (m, 4H), 7.83 (d, J=8.9 Hz, 1H), 7.67-7.64 (m, 1H), 7.52 (s, 1H), 7.37-7.35 (m, 1H), 4.04 (t, J=6.8 Hz, 2H), 2.84 (t, J=7.3 Hz, 2H), 2.59-2.56 (m, 2H), 2.40 (d, J=4.4 Hz, 3H).

MS (ESI⁺, m/z): 461.1 [M+H]⁺

### Example 46: 3-(3-fluoroazetidin-1-yl)-N-methyl-1-(4-(trifluoromethyl)phenyl)-1H-indole-5-sulfonamide

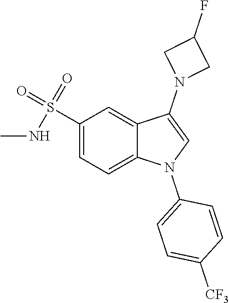 [SMILES: CNS(=O)(=O)c1ccc2c(c1)c(N1CC(F)C1)cn2-c1ccc(C(F)(F)F)cc1]

3-bromo-N-methyl-1-(4-(trifluoromethyl)phenyl)-1H-indole-5-sulfonamide (200 mg, 0.46 mmol) obtained in [Process-6] of Example 1 above was dissolved in 4 mL of 1,4-dioxane, followed by addition of 3-fluoroazetidine hydrochloride (63 mg, 0.55 mmol), tris(dibenzylideneacetone)dipalladium(0) ([Pd₂ (dba)₃], (21 mg, 0.02 mmol), xantphos (40 mg, 0.06 mmol) and cesium carbonate (455 mg, 1.38 mmol) thereto respectively. The reaction solution was stirred at 110° C. for 4 hours to complete the reaction, 10 ml of water was added thereto, and extraction was performed thereon three times with ethyl acetate. The organic layer thus obtained was dried over anhydrous sodium sulfate, filtered under reduced pressure, and the filtered organic layer was concentrated under reduced pressure. The obtained residue was purified by MPLC (ethyl acetate:hexane=1:3 (v/v)) to obtain 10 mg (5% yield) of the title compound.

¹H-NMR (300 MHz, DMSO-d₆): δ 7.94-7.93 (m, 3H), 7.91-7.79 (m, 3H), 7.63-7.59 (m, 1H), 7.36 (s, 1H), 5.64-5.40 (m, 1H), 4.37-4.25 (m, 2H), 4.06-3.93 (m, 2H), 2.40-2.38 (m, 3H).

MS (ESI⁺, m/z): 428.1 [M+H]⁺

### Example 47: N,N-dimethyl-2-(4-(5-(N-methylsulfamoyl)-1-(4-(trifluoromethyl)phenyl)-1H-indol-3-yl)-1H-imidazole-1-yl) acetamide

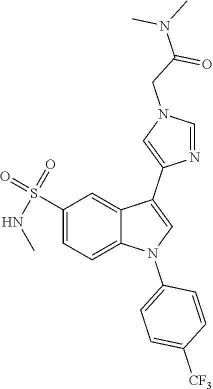 [SMILES: CNS(=O)(=O)c1ccc2c(c1)c(-c1cn(CC(=O)N(C)C)cn1)cn2-c1ccc(C(F)(F)F)cc1]

The procedure of Example 1 was repeated except for the use of N,N-dimethyl-2-(4-(tributylstannyl)-1H-imidazol-1-yl) acetamide (326 mg, 0.74 mmol) instead of tributyl-(3-methylpyrazin-2-yl)stannane in [Process-7] of Example 1 above, to obtain 50 mg (27% yield) of the title compound.

¹H-NMR (300 MHz, DMSO-d₆): δ 8.70 (d, J=1.5 Hz, 1H), 8.16 (s, 1H), 8.00-7.92 (m, 4H), 7.85-7.82 (m, 1H), 7.68 (s, 1H), 7.68-7.64 (m, 1H), 7.49 (s, 1H), 7.36-7.35 (m, 1H), 5.09 (s, 2H), 3.06 (s, 3H), 2.89 (s, 3H), 2.41 (d, J=5.0 Hz, 3H).

MS (ESI⁺, m/z): 506.1 [M+H]⁺

### Example 48: N-methyl-3-(1-(2-(methylsulfonyl)ethyl)-1H-imidazol-4-yl)-1-(4-(trifluoromethyl)phenyl)-1H-indole-5-sulfonamide

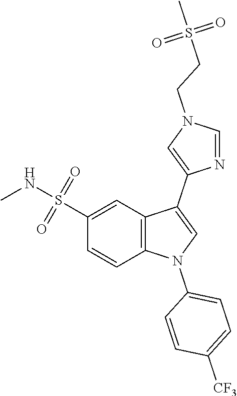 [SMILES: CNS(=O)(=O)c1ccc2c(c1)c(-c1cn(CCS(C)(=O)=O)cn1)cn2-c1ccc(C(F)(F)F)cc1]

The procedure of Example 1 was repeated except for the use of 1-(2-(methylsulfonyl)ethyl)-4-(tributylstannyl)-1H-imidazole (510 mg, 1.10 mmol) instead of tributyl-(3-methylpyrazin-2-yl)stannane in [Process-7] of Example 1 above, to obtain 65 mg (21% yield) of the title compound.

¹H-NMR (300 MHz, DMSO-de): δ 8.70 (d, J=1.4 Hz, 1H), 8.22 (s, 1H), 8.19-7.95 (m, 4H), 7.92-7.82 (m, 2H), 7.75-7.65 (m, 2H), 7.38-7.33 (m, 1H), 4.52 (t, J=7.0 Hz, 2H), 3.77 (t, J=7.4 Hz, 2H), 2.96 (s, 3H), 2.41 (d, J=5.0 Hz, 3H).

MS (ESI⁺, m/z): 527.1 [M+H]⁺

### Experimental Example 1: TEAD Reporter Activity Inhibition Test

The ability of the synthetic compounds to inhibit TEAD target gene transcription was measured. This evaluation method measures the transcriptional activity of TEAD by measuring the luciferase luminescence expressed when TEAD binds to a target gene and activates transcription using the MCF7 cell line (BPS Bioscience, Inc., USA) in which a firefly luciferase reporter gene has been introduced into the specific binding structure of TEAD, GTIIC (5′-ACATTCCA-3′). Cell lines were cultured in MEM medium added with 10% FBS, 1% Penicillin/Streptomycin, 1% non-essential amino acids, 10 μg/ml insulin, and 400 μg/ml Geneticin, and Geneticin was excluded when testing for inhibition of TEAD reporter activity. 4×10+ cells/100 μl were dispensed into a white 96-well plate and cultured for 6 hours. 50 μl of the test compound diluted to 3× concentration was mixed into each well. After 24 hours of cell culture, the luciferase signal was measured by luminescence using the ONE-Glo luciferase assay system (Promega, E6120) following the manufacturer's protocol. The 50% inhibition value (IC₅₀) for TEAD transcriptional activity was calculated using GraphPad Prism 9.

If the IC₅₀ value is less than 100 nM, it is indicated as A, if it is at least 100 nM but less than 500 nM, it is indicated as B, and if it is 500 nM or more, it is indicated as C.

TABLE 1

| Synthetic | |
|---|---|
| compound | IC₅₀, nM |
| 1 | C |
| 2 | A |
| 3 | B |
| 4 | B |
| 5 | B |
| 6 | C |
| 7 | B |
| 8 | A |
| 9 | B |
| 10 | A |
| 11 | A |
| 12 | B |
| 13 | A |
| 14 | B |
| 15 | B |
| 16 | A |
| 17 | B |
| 18 | C |
| 19 | C |
| 20 | A |
| 21 | B |
| 22 | C |
| 23 | A |
| 24 | B |
| 25 | A |
| 26 | C |
| 27 | A |
| 28 | A |
| 29 | B |
| 30 | A |
| 31 | A |
| 32 | A |
| 33 | B |
| 34 | B |
| 35 | B |
| 36 | B |
| 37 | A |
| 38 | A |
| 39 | B |
| 40 | A |
| 41 | B |
| 42 | A |
| 43 | A |
| 44 | A |
| 45 | A |
| 46 | A |
| 47 | C |
| 48 | B |

### Experimental Example 2: Cell Growth Inhibition Test

The synthetic compound was confirmed to inhibit cell growth in NCI-H226 cells. NCI-H226 is a mesothelioma cancer cell line lacking the NF2 gene, and was cultured in RPMI 1640 medium supplemented added with 10% FBS and 1% Penicillin/Streptomycin. Cultured cells were dispensed into 96-well plates at 0.7×10³ cells/100 μl and cultured for 24 hours, then 100 μl of the test compound diluted to a 2× concentration were mixed into each well and cultured for 6 days. The SRB test method was used to measure cell growth inhibition, and the 50% inhibition value (GI₅₀) for cell growth by the compound was calculated using GraphPad Prism 9.

If the GI₅₀ value is less than 100 nM, it is indicated as A, and if it is 100 nM or more, it is indicated as B.

TABLE 2

| Synthetic | |
|---|---|
| compound | GI₅₀, nM |
| 2 | A |
| 8 | A |
| 10 | A |
| 11 | A |
| 13 | B |
| 16 | A |
| 20 | A |
| 23 | A |
| 28 | A |
| 31 | A |
| 32 | A |
| 38 | A |
| 42 | A |
| 43 | A |
| 44 | A |

So far, the present disclosure has been examined focusing on its specific embodiments. A person skilled in the art to which the present disclosure pertains may understand that the present disclosure may be implemented in a modified form without departing from the essential characteristics of the present disclosure. Therefore, the disclosed embodiments should be considered from an illustrative perspective rather than a limiting one. The scope of the present disclosure is indicated in the claims rather than the foregoing description, and all differences within the equivalent scope should be construed as being included in the present disclosure.

1. A compound selected from compounds of Formula 1 below, enantiomers, diastereomers, solvates and hydrates thereof, and pharmaceutically acceptable salts thereof: 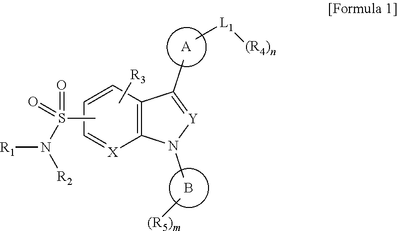
wherein, in Formula 1 above,
R₁ and R₂ are each independently hydrogen, C₁₋₆alkyl, C₂₋₆alkenyl, C₃₋₆carbocyclyl, or haloC₁₋₆alkyl;
R₃ is hydrogen, halogen, C₁₋₆alkyl, haloC₁₋₆alkyl, C₁₋₆alkoxy, or cyano;
\textcircled{A} is carbocyclyl or heterocyclyl;
\textcircled{B} is C₆₋₁₀aryl or C₄₋₁₀heteroaryl;
L₁ is absent, bonded, or C₁₋₃alkylene, or C₁₋₃alkylene substituted with a halogen;
each R₄ and each R₅ are independently hydrogen, halogen, cyano, amino, C₁₋₆alkyl, haloC₁₋₆alkyl, C₁₋₆alkoxy, C₁₋₆alkoxyalkyl, haloC₁₋₆alkoxy, mono-(C₁₋₃alkyl)-substituted carbamoyl (—(CO)—NH(C₁₋₃alkyl)), di-(C₁₋₃alkyl)-substituted carbamoyl (—(CO)—N(C₁₋₃alkyl) 2), C₁₋₃alkylsulfinyl (—(SO)—(C₁₋₃alkyl)), C₁₋₃alkylsulfonyl (—SO₂—(C₁₋₃alkyl)), substituted or unsubstituted C₃₋₆carbocyclyl, substituted or unsubstituted C₆₋₁₀aryl, substituted or unsubstituted C₂₋₆heterocyclyl, substituted or unsubstituted C₄₋₁₀heteroaryl;
X and Y are each independently —C— or —N—; and
m and n are each independently an integer from 0 to 3.

2. The compound of claim 1, wherein \textcircled{A} is C₆₋₁₀ aryl, C₁₋₁₀ heteroaryl, C₆₋₁₄ fused heteroaryl, or C₂₋₆ heterocyclyl,
wherein C₁₋₁₀heteroaryl, C₆₋₁₄ fused heteroaryl, or C₂₋₆heterocyclyl comprises 1 to 4 heteroatoms each independently selected from N, O, and S.

3. The compound of claim 1, wherein \textcircled{A} is a phenyl group, a pyridinyl group, a pyrazinyl group, a pyrazolyl group, an imidazolyl group, a thiophenyl group, a furanyl group, an oxazole group, an azetidinyl group, or

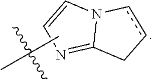

4. The compound of claim 1, wherein \textcircled{B} is a phenyl group or a pyridinyl group.

5. The compound of claim 1, wherein
R₁ and R₂ are each independently hydrogen, C₁₋₆alkyl, C₂₋₆alkenyl, C₃₋₆cycloalkyl, or haloC₁₋₆alkyl.

6. The compound of claim 1, wherein L₁ is a bond;
each of R₄ and each of R₅ are each independently hydrogen, halogen, cyano, C₁₋₆alkyl, haloC₁₋₆alkyl, C₁₋₆alkoxy, C₁₋₆alkoxyalkyl, haloC₁₋₆alkoxy, mono-(C₁₋₃alkyl)-substituted carbamoyl (—(CO)—NH(C₁₋₃alkyl)), di-(C₁₋₃alkyl)-substituted carbamoyl (—(CO)—N(C₁₋₃alkyl)₂), C₁₋₃alkylsulfonyl (—SO₂—(C₁₋₃alkyl)), substituted or unsubstituted C₃₋₆cycloalkyl, substituted or unsubstituted C₆₋₁₀aryl, or substituted or unsubstituted C₂₋₆heterocycloalkyl.

7. The compound of claim 1, wherein L₁ is C₁₋₃alkylene;
each of R₄ and each of R₅ are each independently hydrogen, halogen, cyano, C₁₋₆alkyl, haloC₁₋₆alkyl, C₁₋₆alkoxy, haloC₁₋₆alkoxy, di-(C₁₋₃alkyl)-substituted carbamoyl (—(CO)—N(C₁₋₃alkyl)₂), C₁₋₃alkylsulfonyl (—SO₂—(C₁₋₃alkyl)), substituted or unsubstituted C₃₋₆cycloalkyl, substituted or unsubstituted C₆₋₁₀aryl, or substituted or unsubstituted C₂₋₆heterocycloalkyl.

8. The compound of claim 1, wherein R₅ is each hydrogen, halogen, cyano, C₁₋₆alkyl, haloC₁₋₆alkyl, C₁₋₆alkoxy, haloC₁₋₆alkoxy, C₃₋₆cycloalkyl, or phenyl, or
C₃₋₆ cycloalkyl or phenyl substituted with any one or more substituents selected from halogen, cyano, C₁₋₆alkyl, haloC₁₋₆alkyl, C₁₋₆alkoxy, and haloC₁₋₆alkoxy.

9. The compound of claim 1, wherein R₅ is each hydrogen, halogen, cyano, methyl, ethyl, propyl, 1-butyl, trifluoromethyl, trifluoromethoxy, cyclopropyl, cyclobutyl, cyclopentyl, cyclohexyl, or phenyl.

10. The compound of claim 1, wherein the compound is selected from the following compounds, enantiomers, diastereomers, solvates and hydrates thereof, and pharmaceutically acceptable salts thereof:
N-methyl-3-(3-methylpyrazin-2-yl)-1-(4-(trifluoromethyl)phenyl)-1H-indole-5-sulfonamide;
N-methyl-3-(1-methyl-1H-imidazol-4-yl)-1-(4-(trifluoromethyl)phenyl)-1H-indole-5-sulfonamide;
N-methyl-3-(1-methyl-1H-pyrazol-3-yl)-1-(4-(trifluoromethyl)phenyl)-1H-indole-5-sulfonamide;
N-methyl-3-(1-methyl-1H-pyrazol-4-yl)-1-(4-(trifluoromethyl)phenyl)-1H-indole-5-sulfonamide;
N-methyl-3-(1-methyl-1H-imidazol-4-yl)-1-(4-(trifluoromethoxy)phenyl)-1H-indole-5-sulfonamide;
3-(1-isopropyl-1H-imidazol-4-yl)-N-methyl-1-(4-(trifluoromethyl)phenyl)-1H-indole-5-sulfonamide;
N-ethyl-3-(1-methyl-1H-imidazol-4-yl)-1-(4-(trifluoromethyl)phenyl)-1H-indole-5-sulfonamide;
N-methyl-3-(pyridin-2-yl)-1-(4-(trifluoromethyl)phenyl)-1H-indole-5-sulfonamide;
N-methyl-3-(5-methylthiophen-2-yl)-1-(4-(trifluoromethyl)phenyl)-1H-indole-5-sulfonamide;
N-methyl-3-(5-methylfuran-2-yl)-1-(4-(trifluoromethyl)phenyl)-1H-indole-5-sulfonamide;
3-(1-ethyl-1H-imidazol-4-yl)-N-methyl-1-(4-(trifluoromethyl)phenyl)-1H-indole-5-sulfonamide;
N-methyl-3-(1-methyl-1H-imidazol-4-yl)-1-(5-(trifluoromethyl)pyridin-2-yl)-1H-indole-5-sulfonamide;
3-(2-fluorophenyl)-N-methyl-1-(4-(trifluoromethyl)phenyl)-1H-indole-5-sulfonamide;
1-(4-chlorophenyl)-N-methyl-3-(1-methyl-1H-imidazol-4-yl)-1H-indole-5-sulfonamide;
N-methyl-3-(pyridin-4-yl)-1-(4-(trifluoromethyl)phenyl)-1H-indole-5-sulfonamide;
3-(1-cyclobutyl-1H-imidazol-4-yl)-N-methyl-1-(4-(trifluoromethyl)phenyl)-1H-indole-5-sulfonamide;
N-methyl-3-phenyl-1-(4-(trifluoromethyl)phenyl)-1H-indole-5-sulfonamide;
1-(4-cyclohexylphenyl)-N-methyl-3-(1-methyl-1H-imidazol-4-yl)-1H-indole-5-sulfonamide;
N,N-dimethyl-3-(1-methyl-1H-imidazol-4-yl)-1-(4-(trifluoromethyl)phenyl)-1H-indole-5-sulfonamide;
3-(3-fluoropyridin-2-yl)-N-methyl-1-(4-(trifluoromethyl)phenyl)-1H-indole-5-sulfonamide;
N-methyl-3-(1-methyl-1H-imidazol-4-yl)-1-(3-(trifluoromethyl)phenyl)-1H-indole-5-sulfonamide;
1-(4-cyanophenyl)-N-methyl-3-(1-methyl-1H-imidazol-4-yl)-1H-indole-5-sulfonamide;
3-(furan-3-yl)-N-methyl-1-(4-(trifluoromethyl)phenyl)-1H-indole-5-sulfonamide;
N-methyl-3-(5-methylfuran-2-yl)-1-phenyl-1H-indole-5-sulfonamide;
3-(2,3-difluorophenyl)-N-methyl-1-(4-(trifluoromethyl)phenyl)-1H-indole-5-sulfonamide;
N-methyl-3-(1-methyl-1H-imidazol-4-yl)-1-phenyl-1H-indole-5-sulfonamide;
1-(3-chloro-4-(trifluoromethyl)phenyl)-N-methyl-3-(1-methyl-1H-imidazol-4-yl)-1H-indole-5-sulfonamide;
3-(1-cyclopropyl-1H-imidazol-4-yl)-N-methyl-1-(4-(trifluoromethyl)phenyl)-1H-indole-5-sulfonamide;
3-(1H-imidazol-4-yl)-N-methyl-1-(4-(trifluoromethyl)phenyl)-1H-indole-5-sulfonamide;
3-(1-(2-fluorobenzyl)-1H-imidazol-4-yl)-N-methyl-1-(4-(trifluoromethyl)phenyl)-1H-indole-5-sulfonamide;
3-(furan-2-yl)-N-methyl-1-(4-(trifluoromethyl)phenyl)-1H-indole-5-sulfonamide;
N-methyl-3-(2-methyloxazol-5-yl)-1-(4-(trifluoromethyl)phenyl)-1H-indole-5-sulfonamide;
N-methyl-3-(1-methyl-1H-imidazol-4-yl)-1-(4-(trifluoromethyl)phenyl)-1H-indole-6-sulfonamide;
N-methyl-3-(5-(trifluoromethyl) furan-2-yl)-1-(4-(trifluoromethyl)phenyl)-1H-indole-5-sulfonamide;
N-methyl-3-(1-(oxetan-3-yl)-1H-imidazol-4-yl)-1-(4-(trifluoromethyl)phenyl)-1H-indole-5-sulfonamide;
3-(5-chlorofuran-2-yl)-N-methyl-1-(4-(trifluoromethyl)phenyl)-1H-indole-5-sulfonamide;
N-methyl-3-(1-(2,2,2-trifluoroethyl)-1H-imidazol-4-yl)-1-(4-(trifluoromethyl)phenyl)-1H-indole-5-sulfonamide;
3-(1-(2-methoxyethyl)-1H-imidazol-4-yl)-N-methyl-1-(4-(trifluoromethyl)phenyl)-1H-indole-5-sulfonamide;
N-methyl-3-(1-methyl-1H-imidazol-4-yl)-1-(p-tolyl)-1H-indole-5-sulfonamide;
1-(4-(t-butyl)phenyl)-N-methyl-3-(1-methyl-1H-imidazol-4-yl)-1H-indole-5-sulfonamide;
N-methyl-3-(oxazol-5-yl)-1-(4-(trifluoromethyl)phenyl)-1H-indole-5-sulfonamide;
3-(1-cyclopropyl-1H-imidazol-4-yl)-1-(2-fluoro-4-(trifluoromethyl)phenyl)-N-methyl-1H-indole-5-sulfonamide;
3-(1-cyclopropyl-1H-imidazol-4-yl)-N-methyl-1-(4-(trifluoromethyl)phenyl)-1H-pyrrolo[2,3-b]pyridin-5-sulfonamide;
3-(1-cyclopropyl-1H-imidazol-4-yl)-N-methyl-1-(4-(trifluoromethyl)phenyl)-1H-indazol-5-sulfonamide;
3-(6,7-dihydro-5H-pyrrolo[1,2-a]imidazol-2-yl)-N-methyl-1-(4-(trifluoromethyl)phenyl)-1H-indole-5-sulfonamide;
3-(3-fluoroazetidin-1-yl)-N-methyl-1-(4-(trifluoromethyl)phenyl)-1H-indole-5-sulfonamide;
N,N-dimethyl-2-(4-(5-(N-methylsulfamoyl)-1-(4-(trifluoromethyl)phenyl)-1H-indol-3-yl)-1H-imidazole-1-yl) acetamide; and
N-methyl-3-(1-(2-(methylsulfonyl)ethyl)-1H-imidazol-4-yl)-1-(4-(trifluoromethyl)phenyl)-1H-indole-5-sulfonamide.

11. A method for treating or preventing a disease caused by transcriptional enhancer associate domain (TEAD) activation in a subject, the method comprising administering to the subject a pharmaceutical composition comprising, as an active ingredient, the compound of claim 1.

12. The method of claim 11, wherein the pharmaceutical composition inhibits Yes associated protein (YAP)-transcriptional enhancer associate domain (TEAD) binding.

13. The method of claim 11, wherein the disease is a cancer or tumor.

14. A pharmaceutical composition comprising a compound of claim 1 and a pharmaceutically acceptable additive.
